(19) United States
(12) Patent Application Publication
(10) Pub. No.: US 2006/0014826 A1
(43) Pub. Date: Jan. 19, 2006

(54) Dioxocyclohexane carboxylic acid phenyl amide derivatives for modulating voltage-gated potassium channels and pharmaceutical compositions containing the same

(75) Inventors: Dimitrios Sarantakis, Newtown, PA (US); James J. Bicksler, Titusville, NJ (US); Jerome C. Wu, Princeton, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/151,060

(22) Filed: Jun. 13, 2005

Related U.S. Application Data
(60) Provisional application No. 60/579,261, filed on Jun. 14, 2004.

(51) Int. Cl. A61K 31/366; A61K 31/16

(52) U.S. Cl. 514/452; 514/613

(57) ABSTRACT

Dioxocyclohexane carboxylic acid phenyl amide derivatives of Formula (I) or a tautomer or a pharmaceutically acceptable salt thereof or both, and pharmaceutical compositions containing the same are provided: 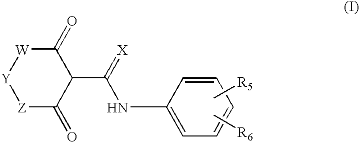 The dioxocyclohexane carboxylic acid phenyl amide derivatives are useful for treating a variety of conditions associated with the abnormal modulation of one or more Kv1.1 voltage-gated potassium channels.

### CROSS-REFERENCE TO RELATED APPLICATIONS

This application claims the benefit under 35 U.S.C. §119(e) to U.S. provisional application 60/579,261, filed Jun. 14, 2004, which is hereby incorporated by reference in its entirety.

### BACKGROUND OF THE INVENTION

The present invention relates to certain dioxocyclohexane carboxylic acid phenyl amide derivatives, to processes for their preparation, and to their use in therapeutic treatments.

Ion channels are transmembrane proteins that regulate the passage of various ions through the membrane. Ion channels are physiologically important, playing essential roles in regulating intracellular levels of various ions and in generating or modulating action potentials in nerve and muscle cells. Passage of ions through ion channels is characterized by selective filtering and by a gating-type mechanism which produces a rapid increase in permeability (Angelides, K. J., et al., J. Biol. Chem., 1981, 258, 11858). Ion channels may be either voltage-gated, implying that current is gated (or regulated) by membrane potential (voltage), or chemically-gated, implying that current is gated primarily by binding of a chemical agent rather than by the membrane potential (Butterworth, J. F., et al., Anesthesiology, 1980, 72, 711). An important characteristic of certain voltage-gated ion channels is inactivation. Soon after opening, these channels close via various mechanisms, forming an inactive channel complex that will not return to its active state until the membrane is repolarized (Miller, C., Science, 1991, 252, 1092).

The ion-conduction pore of these channels is composed of heterotetramers of different, but structurally-related, α-subunits. Each α-subunit consists of an approximately 600-amino-acid polypeptide which possesses six membrane-spanning α-helices (Kolb, 1990; Shi, G., et al., Neuron, 1996, 16, 843). The α-subunits of mammalian voltage-gated potassium channels are currently divided into four related gene-families, based on homology with the corresponding gene families originally derived from work with Drosophila melanogaster. These four groups are the Kv1 (Kv1.1-1.8, the so called Shaker family), Kv2 (Kv2.1-2.2, the Shab family), Kv3 (Kv3.1-3.4, the Shaw family) and Kv4 (Kv4.1-4.1, the Shal family) gene subfamilies (for a review, see Chandy, K. G. and Gutman, G. A., in Handbook of Receptors and Channels: Ligand and Volgate-Gated Ion Channels, CRC Press, pages 1 to 71, 1995 and references cited within.

In some cases, each α-subunit is closely associated with a β-subunit which does not participate in ion conductance directly but can regulate the activity of the channel (for reviews, see: Xu, J. and Li, M., Trends Cardiovasc. Med., 1998, 8, 229; Pongs, O., et al., Ann. N. Y. Acad. Sci., 1999, 868, 344). The known β-subunits have been assigned to three classes, Kvβ1-3, and several splice variants of Kvβ1 have also been described. One well-documented activity of the Kvβ1 subunit is to confer rapid inactivation on the current-conducting α-subunit, which normally displays slow inactivation in the absence of the β-subunit (Rettig, J., et al., Nature, 1994, 369, 289). As in the Drosophila-derived Shaker family, the inactivation conferred by mammalian Kvβ1 involves the N-type “ball-and-chain” mechanism (Zagotta, W. N., et al., Science, 1990, 250, 568; Isacoff, E. Y., et al., Nature, 1991, 353, 86). Thus, in this role Kvβ1 acts as a switch, shutting off the voltage-gated potassium channel once it has performed its function of repolarizing the membrane.

Voltage-gated potassium (Kᵥ) channels participate in several cellular processes. In excitable tissues, these ion channels play an essential role in establishing the resting membrane potential and in modulating the frequency and duration of the action potential (Hille, B., Ionic Channels of Excitable Membranes, Sunderland, Mass., 1992). In nonexcitable cells, they are involved in cell volume regulation, hormone secretion, oxygen sensing and cell proliferation (Kolb., H. A., Rev. Physiol. Biochem. Pharmacol., 1990, 115, 51). Thus, Kv channels are key regulators of neuronal excitability and their dysfunction is believed to be associated with a variety of abnormal conditions or diseases.

For example, implications for a role of Kv1.1 in epilepsy come from varied sources (for a review, see Rho, J. M., Dev. Neurosci., 1999, 21, 320). Kv1.1 genes are richly expressed in brain regions which are susceptible to epileptic seizure, such as hippocampus and neocortex. Kv1.1 potassium channels have been shown to play a role in epilepsy based on recent cloning experiments where deletion of the Kv1.1 potassium channel in mice causes epilepsy (Smart, S. L., et al., Neuron, 1998, 20, 809). In vitro, tissue manipulations using material from these animals which only marginally increase excitability in normal tissue (e.g., raising extracellular potassium or treatment with the GABA_{A} antagonist bicuculine) result in synchronous burst discharges and long-lasting depolarizations in hippocampal CA3 pyramidal neurons. Furthermore, mutations in human genes thought to correspond to Kv1.1 result in hyperexcitable phenotypes, including cases of episodic ataxia and myokymia (Browne, D. L., et al., Nat. Genet., 1994, 8, 136) as well as benign neonatal convulsions, an autosomal dominant form of early-onset epilepsy (Biervert, C., et al., Science, 1998, 279, 403; Charlier, C., et al., Nat. Genet., 1998, 18, 53; Singh, N. A., et al., Nat. Genet., 1998, 18, 25). Additionally, reports of epilepsy in family members affected by the Kv1.1 mutation and diagnosed with episodic ataxia and myokymia have appeared (Zuberi, S. M., et al., Epilepsia, 1997, 38 (supp. 3), 104; Zuberi, S. M., et al., Brain, 1999, 122, 817), correlating the Kv1.1 gene mutation with epilepsy.

Thus, the available evidence suggests that a decrease in Kv1.1 function may act as a mitigating factor in neuronal hyperexcitable disease states, such as epilepsy. Other disease states or conditions affected by neuronal hyperexcitability include for example episodic ataxia, myokymia, neonatalconvulsions, cerebral ischemia, cerebral palsy, stroke, traumatic brain injury, traumatic spinal cord injury, asphyxia, anoxia or prolonged cardiac surgery.

It has also been shown that potassium ion channel openers also play a role in the release and/or regulation of glutamate in mammals (Zini, S. et al., Neuroscience Letters, 1993, 153:202-205). Thus, it is believed compounds that inhibit the inactivation of Kv1.1 will be useful for treating conditions associated with the abnormal release of glutamate including for example hypoglycemia or diseases associated with glutamate release such as Parkinson's disease, Huntingdon's disease, Alzheimer's disease, amyotrophic lateral sclerosis, or AIDS related dementia or combinations thereof.

Thus it would be desirable to find agents that activate Kv1.1 currents or inhibit the inactivation of Kv1.1 currents in mammals. Moreover, as it has been shown that Kv1.1 α-subunits associate and co-localize with Kvβ1 in seizure-sensitive brain regions (Rhodes, K. J., et al., J. Neurosci., 1997, 17, 8246), it would be desirable to find agents that activate or inhibit the inactivation of Kv1.1 potassium channel currents that are associated with Kvβ1.

### SUMMARY OF THE INVENTION

The present invention relates to a method for treating one or more conditions in a mammal associated with the abnormal inactivation (including abnormally decreased activation) of one or more Kv1.1 voltage-gated potassium channels. The method includes administering to a mammal a pharmaceutically effective amount of at least one compound of formula I, or a tautomer or a pharmaceutically acceptable salt thereof or both: 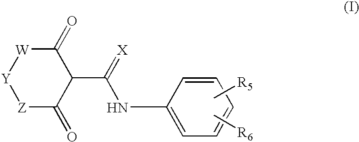
where:

Y is CR₁R₂, S or O;

W is CR₉R₁₀ or O;

Z is CR₃R₄ or O; with the proviso that when Y is O or S, W is CR₉R₁₀ and Z is CR₃R₄, and when W or Z or both are O, Y is CR₁R₂;

X is O or S;

R₁, R₂, R₃, R₄, R₉, and R₁₀ are independently selected from hydrogen, a halogen atom, a hydroxyl group, a C₁ to C₆ alkyl group, a C₁ to C₆ alkoxy group, a C₁ to C₁₂ alkoxyalkyl group a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, or alkanesulfonyl; or two of R₁, R₂, R₃, R₄, R₉, or R₁₀, attached to the same carbon atom or adjacent carbon atoms, may form together with the carbon atom or carbon atoms, in spiro or fused form, a carbocyclic or heterocyclic ring having 3 to 8 carbon atoms and zero to two heteroatoms independently selected from N, O, or S;

R₅ and R₆ are independently selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl; and

R₇ and R₈ are independently selected from hydrogen or a C₁ to C₆ alkyl group;

wherein any moiety containing an alkyl, alkenyl, carbocyclic, heterocyclic, cycloalkyl, heterocycloalkyl, aryl, or heteroaryl group may optionally be substituted with one to three substituents independently selected from a halogen atom, CN, NO₂ or hydroxyl group, a C₁-C₆ alkyl group, a C₁-C₆ alkoxy group, a C₁-C₄ perhalogenated alkyl group or C₁-C₄ perhalogenated alkoxy group.

In another embodiment of the present invention, a method for treating one or more conditions associated with neuronal hyperexcitability is provided that includes administering a pharmaceutically effective amount of at least one compound of formula (I) or a tautomer or a pharmaceutically acceptable salt thereof or both. Conditions related to neuronal hyperexcitability include convulsions, epilepsy, episodic ataxia, myokymia, neonatal convulsions, cerebral ischemia, cerebral palsy, stroke, traumatic brain injury, traumatic spinal cord injury, asphyxia, anoxia or prolonged cardiac surgery.

In another embodiment of the present invention a method for treating one or more conditions associated with the release of glutamate is provided that includes administering a pharmaceutically effective amount of at least one compound of formula (I) or a tautomer or a pharmaceutically acceptable salt thereof, or both. Conditions related to the release of glutamate include pain, Alzheimer's disease, Parkinson's disease, hypoglycemia, Huntingdon's disease, amyotrophic lateral sclerosis, or AIDS related dementia.

In yet another embodiment of the present invention, a method is provided for treating an anxiety disorder in a mammal that includes administering a pharmaceutically effective amount of at least one compound of formula (I) or a tautomer or a pharmaceutically acceptable salt thereof, or both.

The present invention also provides pharmaceutical compositions containing a pharmaceutically effective amount of at least one compound of formula (I) or a tautomer or a pharmaceutically acceptable salt thereof or both and at least one pharmaceutically acceptable carrier.

In yet another embodiment, the present invention also provides compounds of formulas (II) through (VII) or a tautomer or a pharmaceutically acceptable salt thereof or both as defined herein.

### DETAILED DESCRIPTION OF THE INVENTION

The present invention relates to methods for treating one or more conditions in a mammal associated with the abnormal inactivation of Kv1.1 voltage-gated potassium channels. In a preferred embodiment, the Kv1.1 voltage-gated potassium channel that is inhibited from inactivation is associated with one or more Kvβ1 subunits. The compounds useful in the present invention, when administered to a mammal in need thereof, preferably facilitate the activation or inhibit the inactivation of one or more Kv1.1 voltage-gated potassium channels. Preferably, the compounds interact with one or more Kvβ1 subunits to inhibit inactivation.

Compounds useful in the present invention include dioxocyclohexane carboxylic acid phenyl amide derivatives of formula I: 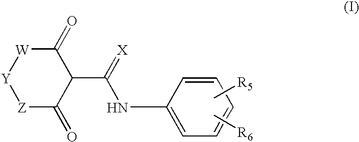
where:

Y is CR₁R₂, S or O;

W is CR₉R₁₀ or O;

Z is CR₃R₄or O; with the proviso that when Y is O or S, W is CR₉R₁₀ and Z is CR₃R₄, and with the proviso that when W or Z or both are O, Y is CR₁R₂;

X is O or S;

R₁, R₂, R₃, R₄, R₉, and R₁₀ are independently selected from hydrogen, a halogen atom, a hydroxyl group, a C₁ to C₆ alkyl group, a C₁ to C₆ alkoxy group, a C₁ to C₁₂ alkoxyalkyl group a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, or alkanesulfonyl; or two of R₁, R₂, R₃, R₄, R₉, or R₁₀, attached to the same carbon atom or adjacent carbon atoms, may form together with the carbon atom or carbon atoms, in spiro or fused form, a carbocyclic or heterocyclic ring having 3 to 8 carbon atoms and zero to two heteroatoms independently selected from N, O, or S;

R₅ and R₆ are independently selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl; and

R₇ and R₈ are independently selected from hydrogen or a C₁ to C₆ alkyl group;

wherein any moiety containing an alkyl, alkenyl, carbocyclic, heterocyclic, cycloalkyl, heterocycloalkyl, aryl, or heteroaryl group may optionally be substituted with one to three substituents independently selected from a halogen atom, CN, NO₂ or hydroxyl group, a C₁-C₆ alkyl group, a C₁-C₆ alkoxy group, a C₁-C₄ perhalogenated alkyl group or C₁-C₄ perhalogenated alkoxy group.

As used herein, unless otherwise indicated, “alkyl” refers to a C₁ to C₆ aliphatic hydrocarbon chain and includes straight or branched chains such as methyl, ethyl, n-propyl, isopropyl, n-butyl, isobutyl, sec-butyl, t-butyl, n-pentyl, isopentyl, neo-pentyl, n-hexyl, isohexyl. Preferably, alkyl groups contain 1 to 4 carbon atoms. “Alkenyl” refers to a C₂ to C₇ aliphatic hydrocarbon chain including straight or branched chains that contains at least one double bond and more preferably 1 to 3 double bonds. Examples of alkenyl groups include vinyl, prop-1-enyl, allyl, methallyl, but-1-enyl, but-2-enyl, but-3-enyl, or 3,3-dimethylbut-1-enyl.

The term “perhalogenated alkyl,” as used herein, refers to a straight or branched aliphatic hydrocarbon chain of 1 to 4 carbon atoms and preferably 1 to 3 carbon atoms, in which all hydrogens are replaced with halogen atoms.

The term “alkoxy,” as used herein, refers to the group R—O— where R is an alkyl group of 1 to 6 carbon atoms.

The term “perhalogenated alkoxy,” as used herein, refers to the group R—O where R is a perhalogenated alkyl group of 1 to 4 carbon atoms.

The term “cycloalkyl,” as used herein, refers to a saturated or partially saturated, hydrocarbon ring containing 3 to 8 carbon atoms and more preferably 5 to 7 carbon atoms, such as cyclopropyl, cyclobutyl, cyclopentyl, cyclohexyl, cycloheptyl, or cyclooctyl. “Heterocycloalkyl” refers to a saturated or partially saturated, hydrocarbon ring having 3 to 8 ring members, preferably, 5 to 7 members, where one to two ring atoms are independently selected from nitrogen, oxygen or sulfur. Exemplary of the heterocycloalkyl rings include the following rings wherein A is NR′, O or S; and R′ is H or an optional substituent described hereinbelow: 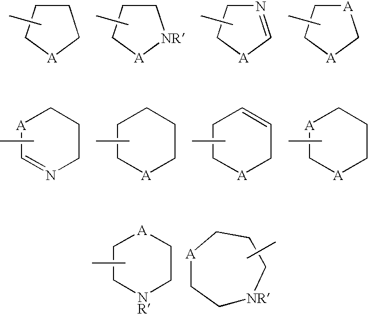
Cycloalkyl or heterocycloalkyl groups may be monocyclic or bicyclic, and more preferably monocyclic. Bicyclic cycloalkyl groups are preferably bridged. “Bridged” refers to a cycloalkyl group that contains at least one carbon-carbon bond between two non-adjacent carbon atoms of the cycloalkyl ring. Moieties containing cycloalkyl or heterocycloalkyl groups may be unsubstituted or substituted on the cycloalkyl or heterocycloalkyl portion of the moiety as described hereinafter.

“Partially saturated” refers to a nonaromatic carbocyclic or heterocyclic group containing at least one double bond and more preferably 1, 2 or 3 double bonds. Preferably, cycloalkyl groups are saturated.

The term “aryl” refers to an aromatic 5- to 7-membered monocarbocyclic ring such as phenyl. “Heteroaryl” refers to an aromatic 5- to 7-membered ring having one or two heteroatoms which independently may be N, O, or S. Examples of heteroaryl include, but are not limited to, thienyl, furyl, pyrrolyl, imidazolyl, pyrazolyl, oxazolyl, isoxazolyl, thiazolyl, isothiazolyl, pyridyl, pyrazinyl, pyrimidinyl, pyridazinyl, indolyl, or indazolyl. Moieties containing Aryl and heteroaryl groups may be unsubstituted or substituted on the aryl or heteroaryl portion of the moiety as described hereinafter.

The term “carbocyclic” refers to a hydrocarbon ring of 3 to 8 carbon atoms which may be saturated, partially saturated, or aromatic and thus includes cycloalkyl and aryl rings. The term “heterocyclic” refers to a ring having 3 to 8 carbon atoms and 1 to 2 heteroatoms independently selected from N, O, or S and thus includes heterocycloalkyl and heteroaryl.

The term “aryloxy,” as used herein, refers to the group Ar—O—, where Ar is an aryl group of 5 to 7 carbon atoms as previously described.

The term “arylalkyl” refers to the group Ar—R— where Ar is an aryl group and R is an alkyl group as previously described.

The term “arylalkyloxy” refers to the group Ar—R—O— where Ar is an aryl group and R is an alkyl group as previously described.

The term “alkoxyalkyl” refers to the group R—O—R₀— where R and R₀ are alkyl groups as previously described.

The term “alkanesulfonyl” refers to the group R—S(O)₂— where R is an alkyl group as previously described.

Halogen means fluorine, chlorine, bromine or iodine.

Unless stated otherwise, any moiety containing an alkyl, alkenyl, carbocyclic, heterocyclic, cycloalkyl, heterocycloalkyl, aryl, or heteroaryl group may be unsubstituted or optionally substituted with one to three substituents as defined hereinafter. For example, alkyl moieties may be halogenated, such as mono- or difluoromethyl or mono- or difluoromethoxy.

The term “substituted” as used herein refers to a moiety, such as an alkyl, alkenyl, carbocyclic, heterocyclic, cycloalkyl, heterocycloalkyl, aryl, or heteroaryl group having from 1 to 3 substituents independently selected from a halogen atom, CN, NO₂, hydroxyl group, a C₁-C₆ alkyl group, a C₁-C₆ alkoxy group, a C₁-C₄ perhalogenated alkyl group or C₁-C₄ perhalogenated alkoxy group. Preferred substituents are halogen atoms, CN, NO₂, hydroxyl group, C₁-C₆ alkyl groups, or C₁-C₆ alkoxy groups.

“mammal” refers to any warm blooded species, such as a human.

“condition associated with the abnormal inactivation of one or more Kv1.1 voltage-gated potassium channels” refers to any abnormal functioning of a mammal or any disease present in a mammal that can at least be partially attributed to abnormal inactivation or abnormally decreased activation of one or more Kv1.1 voltage-gated potassium channels. Examples of conditions that are associated with the inactivation of Kv1.1 voltage-gated potassium channel are described in further detail hereinafter.

“treating” means partially or completely alleviating, inhibiting, preventing and/or ameliorating the condition. For example, “treating” as used herein includes partially or completely alleviating, inhibiting, preventing and/or ameliorating epilepsy.

“Kv1.1 voltage-gated potassium channel” refers to a voltage-gated potassium channel containing at least one Kv1.1 α subunit.

“Kv1.1/Kvβ1” refers to a voltage gated potassium channel that contains at least one Kv1.1 α subunit and that is associated with at least one Kvβ1 subunit.

“Inactivation” refers to the closing of the potassium channel by any mechanism such as by N-type inactivation. “Activation” refers to the potassium channel being open so that current can flow through. Types of inactivation are discussed for example in Hille, B., Ionic Channels of Excitable Membranes, Sunderland, Mass., 1992, which is hereby incorporated by reference in its entirety.

“Pharmaceutically acceptable” refers to a substance that is acceptable for use in pharmaceutical applications from a toxicological perspective and does not adversely interact with the active ingredient.

Compounds useful in the present invention also include all tautomers of formula (I), all pharmaceutically acceptable salts of formula (I), or both (i.e., pharmaceutically acceptable salts of tautomers of formula (I)). Examples of tautomers of formula (I) include:

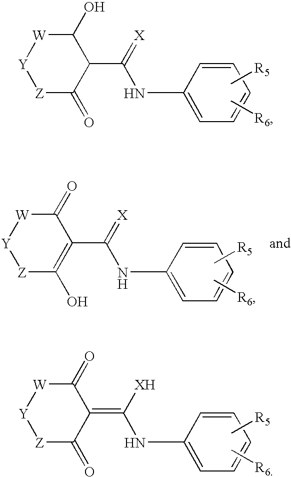

By “pharmaceutically acceptable salt”, it is meant any compound formed by the addition of a pharmaceutically acceptable inorganic or organic base and a compound of formula (I) to form the corresponding salt, or if a basic substituent (e.g., an amino group) is present on the compound of formula (I), any compound formed by the addition of a pharmaceutically acceptable organic or inorganic acid and a compound of formula (I). Examples of pharmaceutically acceptable inorganic or organic bases include alkali metal or alkaline earth metal hydroxides, ammonia, ammonium hydroxide, or basic amines. Examples of organic and inorganic acids include for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids. Examples of compounds useful in this invention include alkali metal (e.g., sodium, potassium, lithium) or alkaline earth metal (e.g., calcium, magnesium) salts of the compounds of formula (I). Examples of salts of formula I formed by the reaction of ammonia, ammonium hydroxide, or a basic amine include the corresponding ammonium; mono-, di-, or trimethylammonium; mono-, di, or triethylammonium; mono-, di-, or tripropylammonium (iso or normal); ethyldimethylammonium; benzyldimethylammonium; cyclohexylammonium; benzylammonium; dibenzylammonium; piperidinium; morpholinium; pyrrolidinium; piperazinium; 1-methylpiperidinium; 1-isopropylpyrrolidinium; 1,4-dimethylpiperazinium; 1-n-butylpiperidinium; 2-methylpiperidinium; 1-ethyl-2-methylpiperidinium; mono-, di-, or triethanolammonium; tris-(hydroxymethyl)methylammonium; or phenylmonoethanolammonium salt.

One skilled in the art will also recognize that the compounds useful in the present invention may contain at least one asymmetric center. While shown without respect to stereochemistry in Formulas I through VII, the compounds useful in the present invention include all optical isomers and diastereoisomers, including all individual isomers, enantiomers, diastereoisomers or mixtures thereof. The compounds useful in the present invention may be prepared as mixtures of the isomers (e.g., racemic) and can be used as such, or may be resolved into the individual isomers.

Where an enantiomer is preferred, it may, in some embodiments be provided substantially free of the corresponding enantiomer. Thus, an enantiomer substantially free of the, corresponding enantiomer refers to a compound which is isolated or separated via separation techniques or prepared free of the corresponding enantiomer. “Substantially free,” as used herein, means that the compound is made up of a significantly greater proportion of one enantiomer. In preferred embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

In one embodiment of the present invention, at least one of R₅ and R₆ is not hydrogen, and in other embodiments at least one of R₅ and R₆ is selected from a halogen atom (preferably F or Cl), a C₁ to C₄ perhalogenated alkyl group (e.g., trifluoromethyl), a C₁ to C₄ perhalogenated alkoxy group (e.g., trifluoromethoxy), or NO₂. In some embodiments, the non-hydrogen R₅ or R₆ substituent is located at the 3, 4, or 5 position of the phenyl ring.

In other embodiments, both R₅ and R₆ are not hydrogen. In some embodiments, R₅ and R₆ are located in the 3,4- or 3,5-positions of the phenyl ring. In this embodiment, preferably R₅ and R₆ are independently selected from a halogen atom (preferably F or Cl), a C₁ to C₄ perhalogenated alkyl group (preferably trifluoromethyl), or a C₁ to C₄ perhalogenated alkoxy group (preferably trifluoromethoxy).

In another embodiment X is oxygen.

Other compounds of the present invention include compounds where Y is CR₁R₂; W is CR₉R₁₀; and Z is CR₃R₄. These compounds have formula II, or tautomers thereof or pharmaceutically acceptable salts thereof, or both: 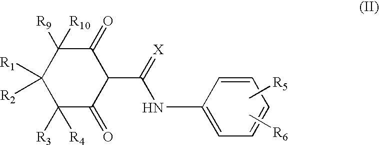
where

R₁, R₂, R₃, R₄, R₉ and R₁₀ are independently selected from hydrogen, a halogen atom, a hydroxyl group, a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, or alkanesulfonyl; or two of R₁, R₂, R₃, R₄, R₉, or R₁₀, attached to the same carbon atom or adjacent carbon atoms, may form together with the carbon atom or carbon atoms, a carbocyclic or heterocyclic ring in spiro or fused form having 4 to 8 carbon atoms and zero to two heteroatoms independently selected from N, O, or S; and

R₅ through R₈ and X are defined as before.

In other embodiments for compounds of formula (II), at least two of R₁, R₂, R₃, R₄, R₉ and R₁₀ are hydrogen, and at least one or two of R₁, R₂, R₃, R₄, R₉ and R₁₀ is not hydrogen. Nonhydrogen substituents are preferably independently selected from hydrogen, a halogen atom, a hydroxyl group, a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group, a C₂ to C₇ alkenyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, or alkanesulfonyl, and more preferably a halogen atom, a hydroxyl group, a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group, or a CO₂R₇.

In other embodiment for compounds of formula (II) at least one of i) R₁ and R₂, ii) R₃ and R₄, or iii) R₉ and R₁₀ are C₁ to C₆ alkyl groups and more preferably C₁ to C₄ alkyl groups, which may be the same or different.

Examples of compounds of formula 11 useful in the methods of the present invention include:
N-(3,4-dichlorophenyl)-4-(isopropyl)-2,6-dioxocyclohexanecarboxamide;
N-(3-chlorophenyl)-4-methyl-2,6-dioxocyclohexanecarboxamide;
N-(3,4-dichlorophenyl)-4,4-dimethyl-2,6-dioxo-cyclohexanecarboxamide;
N-(3-trifluoromethylphenyl)-4,4-dimethyl-2,6-dioxocyclohexanecarboxamide;
N-(3,5-bis-trifluoromethylphenyl)-4,4-dimethyl-2,6-dioxocyclohexanecarboxamide;
N-(4-trifluoromethylphenyl)-4,4-dimethyl-2,6-dioxocyclohexanecarboxamide;
N-(4-fluorophenyl)-4,4-dimethyl-2,6-dioxocyclohexanecarboxamide;
N-(3-trifluoromethoxyphenyl)-4,4-dimethyl-2,6-dioxocyclohexanecarboxamide;
N-(3,4-difluorophenyl)-4,4-dimethyl-2,6-dioxo-cyclohexanecarboxamide;
N-[4-chloro-2-(trifluoromethyl)phenyl]-4,4-dimethyl-2,6-dioxocyclohexanecarboxamide;
4,4-dimethyl-N-(2-nitrophenyl)-2,6-dioxocyclohexanecarboxamide;
4,4-dimethyl-N-(4-nitrophenyl)-2,6-dioxocyclohexanecarboxamide;
N-[4-chloro-3-(trifluoromethyl)phenyl]-4,4-dimethyl-2,6-dioxocyclohexanecarboxamide;
N-(3-chlorophenyl)-4,4-dimethyl-2,6-dioxocyclohexanecarboxamide;
N-(3,4-dichlorophenyl)-4,4-dimethyl-2,6-dioxocyclohexanecarboxamide;
N-[4-chloro-3-(trifluoromethyl)phenyl]-4,4-dimethyl-2,6-dioxocyclohexanecarboxamide;
N-(3-chloro-phenyl)-3,3-dimethyl-2,6-dioxocyclohexanecarboxamide;
N-(3,4-dichlorophenyl)-3,3-dimethyl-2,6-dioxocyclohexanecarboxamide;
N-(3-trifluoromethylphenyl)-3,3-dimethyl-2,6-dioxocyclohexanecarboxamide;
N-(3,5-bis-trifluoromethylphenyl)-3,3-dimethyl-2,6-dioxocyclohexanecarboxamide;
N-(4-trifluoromethylphenyl)-3,3-dimethyl-2,6-dioxocyclohexanecarboxamide;
N-(4-fluorophenyl)-3,3-dimethyl-2,6-dioxocyclohexanecarboxamide;
N-(3-trifluoromethoxyphenyl)-3,3-dimethyl-2,6-dioxocyclohexanecarboxamide;
N-(3,4-difluorophenyl)-3,3-dimethyl-2,6-dioxocyclohexanecarboxamide;
N-(3-trifluoromethylphenyl)-4-ethyl-4-methyl-2,6-dioxocyclohexanecarboxamide;
N-(4-chloro-3-trifluoromethylphenyl)-4-ethyl-4-methyl-2,6-dioxocyclohexanecarboxamide;
N-(3,4-dichlorophenyl)-4-ethyl-4-methyl-2,6-dioxocyclohexanecarboxamide;
N-(3-chloro-4-fluorophenyl)-4-ethyl-4-methyl-2,6-dioxocyclohexanecarboxamide;
N-(4-trifluoromethylphenyl)-4-ethyl-4-methyl-2,6-dioxocyclohexanecarboxamide;
N-(3,5-bis-trifluoromethylphenyl)-4-ethyl-4-methyl-2,6-dioxocyclohexanecarboxamide;
3-Fluoro-4,4-dimethyl-2,6-dioxo-cyclohexanecarboxylic acid(4-trifluoromethyl-phenyl)-amide;
3-Fluoro-4,4-dimethyl-2,6-dioxo-cyclohexanecarboxylic acid(3,5-dichloro-phenyl)-amide;
3-Fluoro-4,4-dimethyl-2,6-dioxo-cyclohexanecarboxylic acid(3,5-difluoro-phenyl)-amide;
4,4-Dimethyl-2,6-dioxo-3-phenyl-cyclohexanecarboxylic acid(3-trifluoromethyl-phenyl)-amide;
3-Methyl-2,6-dioxo-3-phenyl-cyclohexanecarboxylic acid(3-trifluoromethyl-phenyl)-amide;
3-Methoxymethyl-4,4-dimethyl-2,6-dioxo-cyclohexanecarboxylic acid(3-trifluoromethyl-phenyl)-amide; or
a tautomer thereof, a pharmaceutically acceptable salt thereof or both.

In another embodiment of the present invention, the compounds useful in the present invention include those of formula II where two of R₁, R₂, R₃, R₄, R₉, or R₁₀, attached to the same carbon atom or adjacent carbon atoms, may form together with the carbon atom or carbon atoms, a carbocyclic or heterocyclic ring in spiro or fused form having 4 to 8 carbon atoms and zero to two heteroatoms independently selected from N, O, or S, and the remaining R₁, R₂, R₃, R₄, R₉, or R₁₀ are selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl.

Examples of such compounds include:

2,4-Dioxo-3-(3-trifluoromethyl-phenylcarbamoyl)-decahydro-naphthalene-1-carboxylic acid ethyl ester or a tautomer thereof or pharmaceutically acceptable salt thereof, or both.

In some embodiments, the two R substituents forming the carbocyclic or heterocyclic ring are attached to the same carbon atom to form a spiro ring and include for example, compounds of formula (III) or a tautomer thereof or pharmaceutically acceptable salt thereof, or both: 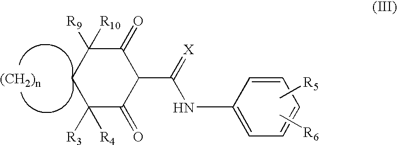
where

the moiety 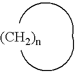
represents a spirocarbocyclic ring;

n is 3 to 5 and more preferably 4 to 5;

R₃, R₄, R₉ and R₁₀ are independently selected from hydrogen, a halogen atom, a hydroxyl group, a C₁ to C₆ alkyl group, a C₁ to C₆ alkoxy group, C₁ to C₆ alkoxyalkyl group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl, where R₅ through R₈ and X are defined as in formula (I).

In some embodiments of formula (III), at least two of R₃, R₄, R₉ and R₁₀ are hydrogen.

Examples of compounds of formula (III) useful in the methods of the present invention include:
7,9-dioxo-spiro[4.5]decane-8-carboxylic acid(4-trifluoromethyl-phenyl)-amide;
7,9-dioxo-spiro[4.5]decane-8-carboxylic acid(3,5-bis-trifluoromethyl-phenyl)-amide;
7,9-dioxo-spiro[4.5]decane-8-carboxylic acid(3-fluoro-phenyl)-amide;
7,9-dioxo-spiro[4.5]decane-8-carboxylic acid(4-chloro-3-trifluoromethyl-phenyl)-amide;
7,9-dioxo-spiro[4.5]decane-8-carboxylic acid(3-trifluoromethoxy-phenyl)-amide;
2,4-dioxo-spiro[5.5]undecane-3-carboxylic acid(4-trifluoromethyl-phenyl)-amide;
2,4-dioxo-spiro[5.5]undecane-3-carboxylic acid(3,5-bis-trifluoromethyl-phenyl)-amide;
2,4-dioxo-spiro[5.5]undecane-3-carboxylic acid(3-chloro-phenyl)-amide;
2,4-dioxo-spiro[5.5]undecane-3-carboxylic acid(3,4-dichloro-phenyl)-amide;
2,4-dioxo-spiro[5.5]undecane-3-carboxylic acid(4-hydroxy-phenyl)-amide; or
a tautomer thereof or a pharmaceutically acceptable salt thereof, or both.

In yet another embodiment, the compounds useful in the present invention include those of formula I, or a tautomer thereof or a pharmaceutically acceptable salt thereof, or both where at least one of W, Y or Z is a non-carbon atom (e.g., Y is O or S, or W, Z or both are O). Examples of compounds where at least one of W, Y or Z is a non-carbon atom include where:

a) W is CR₉R₁₀, Z is CR₃R₄, and Y is oxygen or sulfur as shown below: 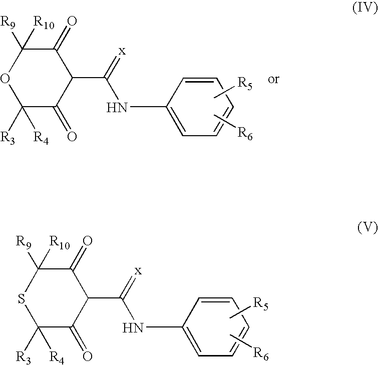
where:

R₃, R₄, R₉ and R₁₀ are independently selected from hydrogen, a halogen atom, a hydroxyl group, a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, alkanesulfonyl; or one of R₉ and R₁₀, or R₃ and R₄ may form together with the carbon atom a saturated or partially saturated carbocyclic or heterocyclic ring in spiro form having 4 to 8 carbon atoms and zero to two heteroatoms independently selected from N, O, or S; and

R₅ through R₈ and X are defined as in formula I.

In some embodiments at least two of R₃, R₄, R₉ and R₁₀ are hydrogen and in other embodiments all are hydrogen.

b) W and Z are oxygen and Y is CR₁R₂ as shown below; 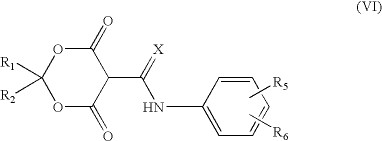
where:

R₁ and R₂ are independently selected from hydrogen, a halogen atom, a hydroxyl group, a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, alkanesulfonyl; or R₁ and R₂ may form together with the carbon atom a saturated or partially saturated carbocyclic or heterocyclic ring in spiro form having 4 to 8 carbon atoms and zero to two heteroatoms independently selected from N, O, or S; and R₅ through R₈ and X are defined as in formula I.

c) one of W and Z is oxygen, and Y is CR₁R₂ as shown below: 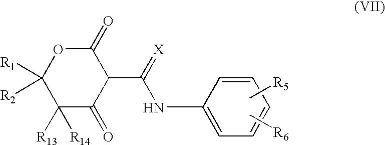
where:

R₁, R₂, R₁₃ and R₁₄, are independently selected from hydrogen, a halogen atom, a hydroxyl group, a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, alkanesulfonyl; or two of R₁, R₂, R₁₃, or R₁₄, at the same carbon atom or adjacent carbon atoms, may form together with the carbon atom or carbon atoms a carbocyclic or heterocyclic ring in spiro or fused form having 4 to 8 carbon atoms and zero to two heteroatoms independently selected from N, O, or S; and R₅ through R₈ and X are defined as in formula I.

Compounds of formula (VII) include where R₁, R₂, R₁₃ and R₁₄, are independently selected from hydrogen, a halogen atom, a hydroxyl group, a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group and more preferably selected from hydrogen or a C₁ to C₆ alkyl group. In other embodiments for compounds of formula (VII): A) at least one of i) R₁ and R₂, or ii) R₁₃ and R₁₄ are not hydrogen and are independently selected from a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group or B) one of i) R₁ and R₂, or ii) R₁₃ and R₁₄, together with the carbon atom to which they are attached form a carbocyclic or heterocyclic ring in spiro form having 3 to 8 carbon atoms and zero to two heteroatoms independently selected from N, O, or S. In one embodiment, at least one of i) R₁ and R₂, or ii) R₁₃ and R₁₄ are the same and are C₁ to C₆ alkyl groups such as methyl, ethyl, propyl or butyl.

Examples of compounds of formula I where at least one of W, Y or Z is a non-carbon atom (e.g., oxygen or sulfur) include:
3,5-dioxo-tetrahydrothiopyran-4-carboxylic acid(4-chloro-3-trifluoromethyl-phenyl)-amide;
3,5-dioxo-tetrahydrothiopyran-4-carboxylic acid(3,4-dichloro-phenyl)-amide;
3,5-dioxo-tetrahydrothiopyran-4-carboxylic acid(3,5-bis-trifluoromethyl-phenyl)-amide;
3,5-dioxo-tetrahydrothiopyran-4-carboxylic acid(4-nitro-phenyl)-amide;
3,5-dioxo-tetrahydrothiopyran-4-carboxylic acid(4-fluoro-3-trifluoromethoxy-phenyl)-amide;
3,5-dioxo-tetrahydrothiopyran-4-carboxylic acid(3-hydroxy-phenyl)-amide;
3,5-dioxo-tetrahydro-pyran-4-carboxylic acid(4-chloro-3-trifluoromethyl-phenyl)-amide;
3,5-dioxo-tetrahydro-pyran-4-carboxylic acid(3,4-dichloro-phenyl)-amide;
3,5-dioxo-tetrahydro-pyran-4-carboxylic acid(4-trifluoromethyl-phenyl)-amide;
3,5-dioxo-tetrahydro-pyran-4-carboxylic acid(3-nitro-phenyl)-amide;
3,5-dioxo-tetrahydro-pyran-4-carboxylic acid(4-trifluoromethoxyl-phenyl)-amide;
3,5-dioxo-tetrahydro-pyran-4-carboxylic acid(4-hydroxy-phenyl)-amide;
N-[4-chloro-3-(trifluoromethyl)phenyl]-6,6-dimethyl-2,4-dioxotetrahydro-2H-pyran-3-carboxamide;
N-[4-(trifluoromethyl)phenyl]-6,6-dimethyl-2,4-dioxotetrahydro-2H-pyran-3-carboxamide;
N-[3-(trifluoromethyl)phenyl]-6,6-dimethyl-2,4-dioxotetrahydro-2H-pyran-3-carboxamide;
N-(3-nitrophenyl)-6,6-dimethyl-2,4-dioxotetrahydro-2H-pyran-3-carboxamide;
N-(3,4-dichlorophenyl)-6,6-dimethyl-2,4-dioxotetrahydro-2H-pyran-3-carboxamide;
N-[3-(trifluoromethyl)phenyl]-5,5-dimethyl-2,4-dioxotetrahydro-2H-pyran-3-carboxamide;
N-[4-(trifluoromethyl)phenyl]-5,5-dimethyl-2,4-dioxotetrahydro-2H-pyran-3-carboxamide;
N-[4-chloro-3-(trifluoromethyl)phenyl]-5,5-dimethyl-2,4-dioxotetrahydro-2H-pyran-3-carboxamide;
7,9-Dioxo-6,10-dioxa-spiro[4.5]decane-8-carbothioic acid(4-chloro-3-trifluoromethyl-phenyl)-amide; or
a tautomer thereof or a pharmaceutically acceptable salt thereof, or both.

The compounds useful in the present invention can be prepared according to techniques well known to those skilled in the art such as disclosed in Chem. Rev., 1999, 99, 1047-1065, and U.S. Pat. Nos. 3,801,630 and U.S. Pat. No. 3,976,785 and GB 1,377,313, the disclosures of which are hereby incorporated by reference in their entireties.

One method for synthesizing compounds of Formula I involves the acylation of a 1,3-cyclic diketones according to the reaction scheme shown below: 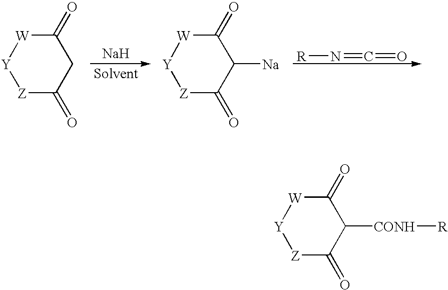
where W, Y, Z, are defined as previously herein and R is the moiety:

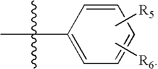

The starting 1,3-cyclic diketone can either be purchased from commercial sources or prepared according to published procedures (See e.g., Chem. Rev. (1999) 99, 1047-1065). The “active methylene” on the 1,3-cyclic diketones is achieved by first forming the sodium salt of the 1,3-cyclic diketone in a suitable solvent such as dimethylformamide (DMF). The salt is then treated with an aryl isocyanate or thioisocyanate as shown above.

Examples of suitable 1,3-diketones that can be used to produce compounds useful in the present invention include:

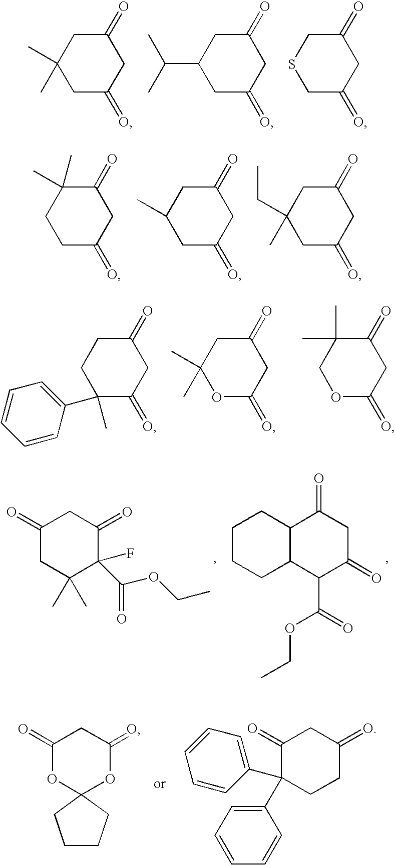

Examples of suitable arylisocyanates and isothiocyanates that can be used as acylating agents in the reaction scheme above include:

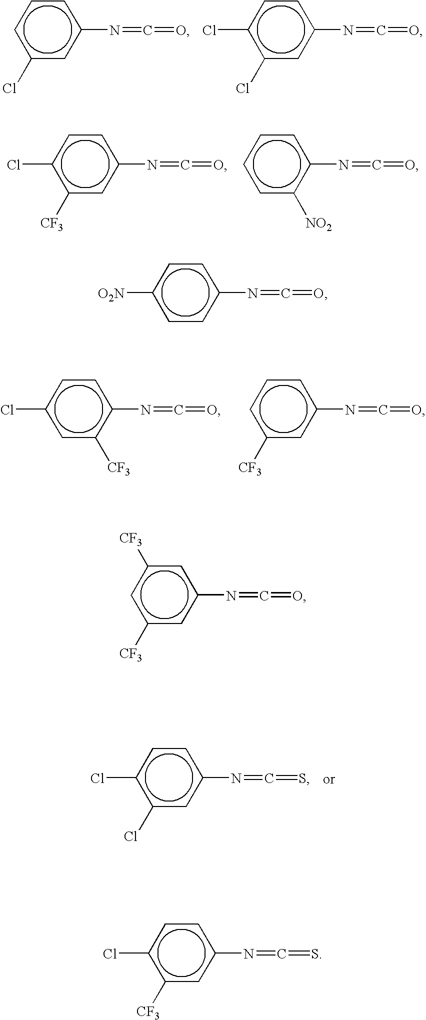

As mentioned previously, the compounds useful in the present invention activate or inhibit the inactivation of Kv1.1 voltage-gated potassium channels in a mammal. The compounds are especially useful for inhibiting the inactivation of Kv1.1/Kvβ1voltage-gated potassium channels. The compounds of formula (I) are preferably useful for inhibiting N-type inactivation of Kv1.1 voltage-gated potassium channels.

Accordingly, the present invention relates to in vitro or in vivo methods of modulating the activity of the Kv1.1 voltage-gated potassium channel. Such methods comprise, in some embodiments of the invention, contacting Kv1.1 voltage-gated potassium channel with a compound of formula (I). In certain embodiments, such methods further comprise monitoring the activity of Kv1.1. In certain embodiments of the invention, the invention relates to methods of modulating the activity of the Kv1.1 voltage-gated potassium channel comprising in vitro or in vivo administration of a pharmaceutically effective amount of one or more compounds of formula (I).

In other embodiments, the invention relates to in vitro or in vivo methods of inhibiting the inactivation of, or activating of the Kv1.1 voltage-gated potassium channel. Such methods comprise, in some embodiments of the invention, contacting Kv1.1 with a compound of formula (I). In certain embodiments, such methods further comprise monitoring the activity of Kv1.1. In certain embodiments of the invention, the invention relates to methods of inhibiting the inactivation of, or activating of the Kv1.1 voltage-gated potassium channel comprising in vitro or in vivo administration of an effective amount of one or more compounds of formula (I).

The compounds of formula (I) are useful for treating a variety of conditions in a mammal associated with the abnormal inactivation of Kv1.1 voltage-gated potassium channels. Conditions that are believed to be associated with the inactivation of voltage gated potassium channels include for example conditions associated with neuronal hyperexcitability, abnormal glutamate regulation, epilepsy, stroke, convulsions, or anxiety disorders, or combinations thereof. In a preferred embodiment of the present invention, the condition treated is epilepsy or stroke.

Conditions associated with neuronal hyperexcitability that can be treated in accordance with the methods of the present invention include for example convulsions, including neonatal convulsions, epilepsy, episodic ataxia, myokymia, cerebral ischemia, cerebral palsy, stroke, traumatic brain injury, traumatic spinal cord injury, asphyxia, anoxia or prolonged cardiac surgery or combinations thereof.

Conditions associated with the abnormal regulation of glutamate include for example hypoglycemia or diseases associated with abnormal glutamate regulation such as Parkinson's disease, Huntingdon's disease, Alzheimer's disease, amyotrophic lateral sclerosis, or AIDS related dementia or combinations thereof.

Another condition associated with the abnormal regulation of glutamate is pain experienced by mammals. For example, the compounds of the present invention may be used for treating acute pain (short duration) or chronic pain (regularly reoccurring or persistent) in mammals. This pain may also be centralized or peripheral.

Examples of pain that can be acute or chronic and that can be treated in accordance with the methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain, or headaches such as migraines or tension headaches, or combinations of these pains. One skilled in the art will recognize that these pains may overlap one another. For example, a pain caused by inflammation may also be visceral or musculoskeletal in nature.

In some embodiments of the present invention the compounds useful in the present invention are administered in mammals to treat chronic pain such as neuropathic pain associated for example with damage to or pathological changes in the peripheral or central nervous systems; cancer pain; visceral pain associated with for example the abdominal, pelvic, and/or perineal regions or pancreatitis; musculoskeletal pain associated with for example the lower or upper back, spine, fibromylagia, temporomandibular joint, or myofascial pain syndrome; bony pain associated with for example bone or joint degenerating disorders such as osteoarthritis, rheumatoid arthritis, or spinal stenosis; headaches such migraine or tension headaches; or pain associated with infections such as HIV or Shingles, sickle cell anemia, autoimmune disorders, multiple sclerosis, or inflammation such as osteoarthritis or rheumatoid arthritis. One skilled in the art will also recognize that the pain treated according to the methods of the present invention may also be related to conditions of hyperalgesia, allodynia, or both. Additionally, the chronic pain may be with or without peripheral or central sensitization.

Additional conditions associated with Kv1.1 inactivation that may be treated in accordance with the methods of the present invention include anxiety disorders such as panic attack, agoraphobia, panic disorder, specific phobia, social phobia, obsessive compulsive disorder, posttraumatic stress disorder, acute stress disorder, generalized anxiety disorder, separation anxiety disorder, substance-induced anxiety disorder, and anxiety disorder not otherwise specified.

Thus the present invention provides methods of treating each of the conditions listed above in a mammal, preferably in a human, the methods comprising administering a pharmaceutically effective amount of a compound of this invention to the mammal in need thereof. The term “administer” or “administering” as used herein means either directly administering a compound useful in the present invention or a pharmaceutical composition containing the compound, or administering the compound or pharmaceutical composition indirectly via a prodrug derivative or analog which will form an equivalent amount of the active compound or substance within the body.

Thus, the present invention includes prodrugs of compounds of Formula I. Prodrug, as used herein, means a compound which is convertible in vivo by metabolic means (e.g. by hydrolysis) to a compound of Formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975).

The dioxocyclohexane carboxylic acid phenyl amide derivatives useful in the present invention can be administered in a variety of ways including for example by oral or parenteral administration such as by intramuscular, intraperitoneal, epidural, intrathecal, intravenous, subcutaneous, intramucosal such as sublingual or intranasal, vaginal, rectal or transdermal administration. In some embodiments of the present invention, the compounds useful in the present invention are administered by oral or intranasal route.

The compounds useful in the present invention are administered in a pharmaceutically effective amount to the mammal needing treatment. As used herein “pharmaceutically effective amount” is at least the minimal amount of the dioxocyclohexane carboxylic acid phenyl amide derivative or a pharmaceutically acceptable salt form thereof, which treats the condition in question in a mammal. The pharmaceutically effective amount will depend on such variables as the particular composition used, the route of administration, the severity of the symptoms, and the particular patient being treated. To determine the pharmaceutically effective amount of the compound to be administered, the physician may, for example, evaluate the effects of a given dioxocyclohexane carboxylic acid phenyl amide derivative in the patient by incrementally increasing the dosage until the desired symptomatic relief level is achieved. The continuing dose regimen may then be modified to achieve the desired result. Based on the results obtained in standard pharmacological tests, projected oral dosages would preferably range from about 0.1 mg/kg to about 1000 mg/kg.

The dioxocyclohexane carboxylic acid phenyl amide derivatives useful in the present invention may be administered neat (i.e., as is) or in a pharmaceutical composition containing at least one pharmaceutically acceptable carrier. Thus, the present invention also provides pharmaceutical compositions containing a pharmaceutically effective amount of at least one compound of formula I or a tautomer thereof or pharmaceutically acceptable salt thereof, or both, and at least one pharmaceutically acceptable carrier. Preferred compounds to be present in the pharmaceutical compositions of the present invention include those compounds previously described. Pharmaceutically acceptable carriers are those that are compatible with the other ingredients in the formulation and biologically acceptable. The pharmaceutical compositions may be administered to a mammal to treat a variety of conditions associated with abnormal inactivation of voltage-gated potassium channels as previously described herein.

Pharmaceutical compositions useful in the present invention may be in any form known to those skilled in the art such as in liquid or solid form. The proportion of ingredients will depend on such factors as the solubility and chemical nature of the compound of formula I and the chosen route of administration. Such compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985).

Pharmaceutical compositions, in addition to containing a pharmaceutically effective amount of one or more dioxocyclohexane carboxylic acid phenyl amide derivatives of the present invention and a pharmaceutically acceptable carrier may include one or more other ingredients known to those skilled in the art for formulating pharmaceutical compositions. Such ingredients include for example, flavoring agents, lubricants, solubilizers, suspending agents, fillers, glidants, compression aids, binders, tablet-disintegrating agents, encapsulating materials, emulsifiers, buffers, preservatives, sweeteners, thickening agents, coloring agents, viscosity regulators, stabilizers or osmo-regulators, or combinations thereof.

Solid pharmaceutical compositions preferably contain one or more solid carriers, and optionally one or more other additives such as flavoring agents, lubricants, solubilizers, suspending agents, fillers, glidants, compression aids, binders or tablet-disintegrating agents or an encapsulating material. One skilled in the art will recognize that some of these other additives also serve as carriers. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes or ion exchange resins, or combinations thereof. In powder pharmaceutical compositions, the carrier is preferably a finely divided solid which is in admixture with the finely divided active ingredient. In tablets, the active ingredient is mixed with a carrier having the necessary compression properties in suitable proportions, and optionally, other additives, and compacted into the desired shape and size. Solid pharmaceutical compositions, such as powders and tablets, preferably contain up to 99% of the active ingredient.

Liquid pharmaceutical compositions preferably contain one or more dioxocyclohexane carboxylic acid phenyl amide derivatives and one or more liquid carriers to form for example solutions, suspensions, emulsions, syrups, elixirs, or pressurized compositions. Pharmaceutically acceptable liquid carriers include for example water, organic solvent, pharmaceutically acceptable oils or fat, or combinations thereof. The pharmaceutical composition can contain other suitable pharmaceutical additives such as solubilizers, emulsifiers, buffers, preservatives, sweeteners, flavoring agents, suspending agents, thickening agents, colors, viscosity regulators, stabilizers or osmo-regulators, or combinations thereof. One skilled in the art will recognize that some of these other additives also serve as carriers.

Examples of liquid carriers suitable for oral or parenteral administration include water (preferably containing additives such as cellulose derivatives such as sodium carboxymethyl cellulose), alcohols or their derivatives (including monohydric alcohols or polyhydric alcohols such as glycols) or oils (e.g., fractionated coconut oil and arachis oil). For parenteral administration the carrier can also be an oily ester such as ethyl oleate and isopropyl myristate. The liquid carrier for pressurized compositions can be halogenated hydrocarbons or other pharmaceutically acceptable propellant.

Liquid pharmaceutical compositions which are sterile solutions or suspensions can be administered parenterally, for example by, intramuscular, intraperitoneal, epidural, intrathecal, intravenous or subcutaneous injection. Pharmaceutical compositions for oral or transmucosal administration may be either in liquid or solid composition form.

The compounds of this invention may be administered rectally or vaginally in the form of a conventional suppository. For administration by intranasal or intrabronchial inhalation or insufflation, the compounds of this invention may be formulated into an aqueous or partially aqueous solution, which can then be utilized in the form of an aerosol. The compounds of this invention may also be administered transdermally through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, such as tablets or capsules. In such form, the composition is sub-divided in unit dose containing appropriate quantities of the active ingredient. The unit dosage forms can be packaged compositions, for example packeted powders, vials, ampoules, pre-filled syringes or sachets containing liquids. The unit dosage form can be, for example, a capsule or tablet itself, or it can be the appropriate number of any such compositions in package form.

Thus, the present invention also provides a pharmaceutical composition in unit dosage form that contains a pharmaceutically effective unit dosage of at least one dioxocyclohexane carboxylic acid phenyl amide derivative of the present invention. As one skilled in the art will recognize, the preferred pharmaceutically effective unit dosage will depend on for example the method of administration. For example, a unit dosage for oral administration preferably ranges from about 0.1 mg to about 1000 mg the dioxocyclohexane carboxylic acid phenyl amide derivatives useful in the present invention.

### EXAMPLES

Compounds of the present invention were prepared and evaluated for inhibiting the inactivation of voltage-gated potassium channels and for inhibiting seizures and anxiety.

The following compounds of formula (I) were prepared. In these examples, all chemicals and intermediates are either commercially available, can be prepared by standard procedures found in the literature, and/or their synthesis is described herein.

### Intermediate 1—4-Ethyl-4-methyl-dihydro-pyran-2,6-dione

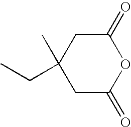

An acetic anhydride solution (15 ml) of 3-ethyl-3-methyl-pentanedioic acid (500 mg, 2.87 mmole) was put under reflux for 3.5 hours. After the reaction was cooled down, the excess acetic anhydride and resulting acetic acid were removed under reduced pressure to give a crude product (440 mg).

### Intermediate 2—3-Ethyl-3-methyl-5-oxo-hexanoic acid

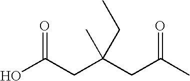

To a THF solution (15 ml) of 4-ethyl-4-methyl-dihydro-pyran-2,6-dione (440 mg), copper iodide (55 mg, 0.29 mmole) was added. The solution was cooled down to −15˜20° C. at which temperature methylmagnesium bromide (3 M solution in ethyl ether, 0.96 ml, 2.87 mmole) was added. The reaction was slowly brought up to room temperature, stirred for 2 hours, and quenched with water. The mixture was acidified with 2 N HCl, and the white solid residue was filtered. The filtrate was extracted with ethyl ether. The organic layer was collected, dried over MgSO₄ and concentrated to give the crude product (554 mg).

### Intermediate 3—3-Ethyl-3-methyl-5-oxo-hexanoic acid methyl ester

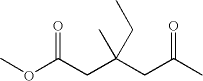

A methanol solution (15 ml) of 3-ethyl-3-methyl-5-oxo-hexanoic acid (554 mg) and concentrated HCl (0.5 ml) was put under reflux overnight. After the reaction was cooled down, methanol was removed under reduced pressure. Water was added, and the mixture was extracted with ethyl ether. The organic layer was collected, dried over MgSO₄ and concentrated. Column chromatography on silica gel eluted with 10% EtOAc/Hexane gave the product in colorless oil (292 mg). The total yield based on the starting material from Intermediate 1 was 54.6%.

### Intermediate 4—5-Ethyl-3-hydroxy-5-methyl-cyclohex-2-enone

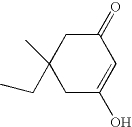

To a THF solution (15 ml) of 3-ethyl-3-methyl-5-oxo-hexanoic acid methyl ester (286 mg, 1.54 mmol), NaH (60% dispersion in mineral oil, 74 mg, 1.85 mmole) was added. The reaction was stirred for 1.5 hours and quenched with water. The mixture was acidified with 1 N HCl and extracted with EtOAc. The organic layer was collected, dried over MgSO₄ and concentrated. The residue was crystallized with Et2O to give the product in white solid (141 mg). Yield: 59.4%.

### Intermediate 5—6,6-Dimethyl-dihydro-pyran-2.4-dione

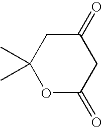

Methyl acetoacetate (5.4 ml, 50.0 mmole), n-BuLi (2.5 M in hexane, 21 ml, 52.5 mmole), and acetone (4.0 ml, 55.0 mmole) were each added 30 minutes apart into THF solution with NaH (60% dispersion in mineral oil, 2.4 g, 60.0 mmole) at 0° C. The mixture was washed with EtOAc, and concentrated HCl was added to the water layer. The acidified water layer was extracted with EtOAc. The organic layer was collected, dried over MgSO₄, and concentrated to give the crude product, which was further crystallized to give a light yellow solid (1.4 g). Yield: 19.7%. MS (+) ESI m/z=143 [M+H]⁺.

Analysis for C₁₇H₁₈F₃NO₃: Calculated: C: 59.14; H: 7.09; N:0, Found: C: 58.51; H: 7.08; N: 0.01.

### Intermediate 6—5,5-Dimethyl-dihydro-pyran-2,4-dione

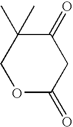

n-Butylithium (2.5 M in hexane, 19.0 ml, 46.7 mmole) was slowly added to a THF solution of diisopropylamine (6.67 ml, 47.6 mmole) at room temperature. After 30 minutes, the solution was cooled down to −78° C. at which THF solution of 3-acetoxy-2,2-dimethyl-propionic acid methyl ester (4.41 g, 23.8 mmole) was added. The reaction was stirred for 2.5 hours at −78° C. before it was quenched with water at room temperature. THF was removed under reduced pressure. The mixture was extracted with EtOAc. The organic layer was collected, washed with water, dried over MgSO₄ and concentrated. Column chromatography on silica gel eluted with 50% EtOAc in hexane give the product. Yield: 58.0%.

### Intermediate 7—1-Fluoro-2,2-dimethyl-4,6-dioxo-cyclohexanecarboxylic acid ethyl ester

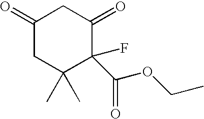

0.8 g (0.345 mole) of sodium metal was stirred in 200 ml of absolute ethanol until dissolved. 3.6 ml (0.317 mole) of mesityl oxide and 5.0 ml (0.317 mole) of diethylfluoromalonate were added. The reaction was refluxed for four hours. The ethanol was removed via a rotoevaporator. The residue was taken up in 200 ml water and washed with ethyl ether. The aqueous layer was acidified with 2N HCl and extracted twice with methylene chloride. The combined methylene chloride extracts were dried over sodium sulfate, filtered and stripped to a yellow oil (6.9 g). The oil was triturated with hexane to afford a white solid (6.5 g-89% yield). MS: (−) EI (M−H)⁻ 229, Elemental Anal. for: C₁₈H₁₉CIFNO5: Calcd.: C, 54.20%; H, 6.82%; N, 0.00%,

Found: C, 52.44%; H,6.27%; N, 0.00%, ¹H-NMR (DMSO-d₆): δ 4.2 (m, 2H); 2.7(d,1H); 2.3(d,1H); 1.2(m, 3H); 1.1(d, 6H).

### Intermediate 8—4,4-Dimethyl-cyclohexane-1,3-dione

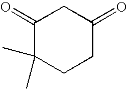

25.0 g (1760 mole) of 2,2 dimethylglutaric anhydride was stirred with 5.66 g (0. 1760 mole) of methanol in an oil bath set at 67° C. for one hour. 13 ml (0.1936 mole) of thionyl chloride was added and the reaction was stirred overnight. Excess thionyl chloride was removed via a rotoevaporator. The residue was vacuum distilled (b.p. 90-106° C.) to afford a 14.5 g mixture of 4-chlorocarbonyl-4-methyl-pentanoic acid methyl ester and 4-chlorocarbonyl-2,2-dimethyl-butyric acid methyl ester (43% yield). 10.0 g (0.519 mole) of this mixture were placed in a 500 ml three neck flask equipped with a dropping funnel, nitrogen bubbler and thermometer. The system was flushed with nitrogen for thirty minutes. 21 ml of 2M dimethyl zinc in toluene was transferred to the dropping funnel via a syringe. The dimethyl zinc was added dropwise to the stirring solution (25 minutes). The reaction was then heated to 45° C. in a water bath and stirred for forty-five minutes. The reaction was cooled to room temperature and 50 ml of sat. NH₄Cl was added carefully (methane gas evolved). The layers were allowed to separate and the aqueous layer was extracted twice with benzene. The combined benzene extracts were washed with sat. NaHCO₃ and dried over sodium sulfate, filtered and stripped to 7.5 g (84% yield) of a colorless oil. 7.8 g (4.0 eq.) of sodium methoxide were suspended in 100 ml of ethyl ether and cooled to 5° C. 2,2-dimethyl-5-oxo-hexanoic acid methyl ester (7.5 g) (0.043 mole) was taken up in 25 ml of ethyl ether and added slowly to the reaction. The reaction was warmed to room temperature and stirred overnight. Cold water (100 ml) was added to the reaction and the ether layer was removed after shaking. The aqueous layer was acidified with 1N HCl and extracted with ethyl acetate. The ethyl acetate layer was dried over sodium sulfate, filtered and stripped to 5.8 g of a colorless oil. The crude product was taken up in 97.5/2.5 methylene chloride/methanol and charged on a Biotage Flash 90 cartridge (silica). The weight of the pure product which was eluted with the methylene chloride/methanol was 3.0 g ( 50% yield). MS: (−) ES (M−H)⁻ 139, Elemental Anal. Calcd. for: C₈H₁₂O₂=0.4 mole H₂O, C, 65.19%; H, 8.75%, N, 0.00%, Found: C, 65.13%; H, 8.20%; N, 0.00%, ¹H-NMR (DMSO-d₆): δ 5.09 (s, 1H); 3.31 (b,1H); 2.32 (m, 2H): 1.69 (t, 2H); 0.99 (s, 6H).

### Intermediate 9—4-Methyl-4-phenyl-cyclohexane-1.3-dione

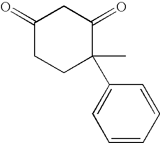

To a stirring suspension of 0.337 g (0.0030 mole) of potassium t-butoxide in 15 ml ethyl ether was added 0.45 g (0.0030 mole) of 3-phenyl-butan-2-one in 10 ml ethyl ether. The reaction was stirred for ten minutes. The reaction was stirred overnight at room temperature. A white precipitate was collected on a filter and washed with ethyl ether. The crude product was dissolved in water, acidified with dilute HCl and extracted (three times) into chloroform. The combined chloroform extracts were dried over sodium sulfate, filtered and stripped to a solid (0.45 g). MS: (−) ES (M−H) 201

### Intermediate 10—2,4-Dioxo-decahydro-naphthalene-1-carboxylic acid ethyl ester

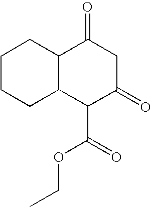

2.66 g of sodium metal (0.115 mole) were stirred in 150 ml absolute ethanol until dissolved. 16 ml (0.1053 mole) of diethyl malonate were added to the reaction. The reaction was stirred for 5 min, then 12.25 ml of 1-acetyl-1-cyclohexane was added. The reaction was refluxed for 4 hrs, then was stirred overnight at room temperature. The volume of ethanol was reduced to half, 200 ml H₂O was added and the reaction was washed with ethyl ether. The aqueous layer was acidified with 2N HCl and extracted into ethyl ether (3 times). Combined ether extracts were dried over Na₂SO₄, filtered and stripped to afford a brown oil, 23.9 gms (95% yield). M.S.: (−)ES [M−H]⁻ 237

### Intermediate 11—6,10-Dioxa-spiro[4.5]decane-7,9-dione

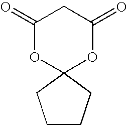

11.4 g (0.1095 mole) of malonic acid was stirred with 25 ml of acetic anhydride and three drops of sulfuric acid for two hours. 9.7 ml (0.1095 mole) of cyclopentanone was added to the reaction. The reaction mixture was stirred overnight at room temperature. The excess acetic anhydride was removed via vacuum pump/rotoevaporator. The residue was triturated with hexane and collected on a filter. Crude crystals were dissolved in ethyl ether. Hexane was added to the ether until brown crystals began to form. The crystals were filtered off and discarded. The ethyl ether-hexane filtrate was allowed to stand until off white crystals formed. They were collected on a sintered glass filter and washed with hexane and dried. The dry weight of the crystals afforded 11.5 g (62% yield) of pure product.

MP=68° C., MS: (−) ES (M−H)⁻ 169, Elemental Anal. for: C₈H₁₀O₄+0.25 mole H₂O: Calcd.: C, 55.01%; H, 6.06%; N, 0.00%, Found: C, 54.75%; H, 5.90%; N, 0.00%, ¹H-NMR (DMSO-d₆): δ 4.0 (s, 2H); 2.1 (m, 4H); 1.7 (m, 4H).

### Intermediate 12—4,4-Diphenyl-cyclohexane-1,3-dione

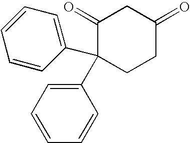

To a stirring suspension of 2.8 g (0.025 mole) of potassium t-butoxide in 50 ml ethyl ether was added 0.53 g (0.025 mole) of 1,1 diphenylacetone in 25 ml ethyl ether. The reaction was stirred for ten minutes; the white slurry became a clear yellow solution. 2.7 ml (0.025 mole) of ethyl acrylate in 50 ml ethyl ether was added. A white precipitate formed immediately. The reaction was stirred overnight at room temperature. The white precipitate was collected on a filter and washed with ethyl ether. The crude product was dissolved in water, acidified with dilute HCl and extracted (three times) into chloroform. The combined chloroform extracts were dried over sodium sulfate, filtered and stripped to a solid (4.1 g). MS: (−) ES (M−H)⁻ 263, Elemental Anal. Calcd. for C₁₈H₁₆O₂+0.5 mole H₂O: C, 79.10%; H, 6.27%; N, 0.00%, Found: C, 79.09%; H, 5.90%; N, 0.02%, 1H-NMR (DMSO-d6) δ 11.13 (b, 1H); 7.27 (m, 6H); 7.10 (m, 4H); 5.38 (s,1H); 2.64 ( m, 2H); (s, 2H).

### Example 1

### N-(3-chlorophenyl)-4,4-dimethyl-2,6-dioxocyclohexanecarboxamide

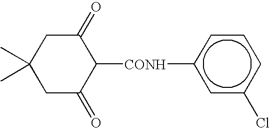

A solution of 5,5-dimethyl cyclohexanedione (dimedone) (1.25 g, 8.9 mmole) in DMF (25 ml) was treated with NaH (1.1 equiv., 0.39 g) and the mixture was stirred for 25 minutes. The cloudy mixture became clear. One equiv. of 3-chlorophenyl isocyanate (1.1 ml) was added and the mixture was stirred overnight at room temperature. The reaction mixture was evaporated to dryness to give a yellow oil which was partitioned between EtOAc and water. The organic phase was washed with water, 1N-aq. HCl, brine and dried to afford a solid, 2.8 g. This crude product was chromatographed (chromd.) through silica gel (elution with DCM-Hexane, 3:1, v/v) to provide 1.5 g of the title compound. M.P:=89-91° C.; MS: (-ESI) (M−H)⁻ 292; TLC: silica gel, Rf=0.6 (DCM); HPLC: C¹⁸-column, 70/30 acetonitrile-water/0.1% H₃PO₄; 99.88%; Elemental Anal: Calcd. for: C₁₅H₁₆CINO₃: C, 61.33%; H, 5.49%; N, 4;77%, Found: C, 61.21%; H, 5.42%; N, 4.70%; ¹H-NMR (DMSO-d₆) δ 7.8 (s, 1H); 7.4 (m, 2H); 7.2 (m,1H); 2.5 (d, 4H); 1.0 (s, 6H).

### Example 2

### N-(3,4-dichlorophenyl)-4,4-dimethyl-2,6-dioxocyclohexanecarboxamide

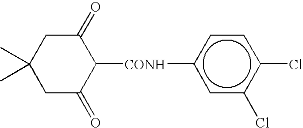

The title compound was prepared according to the procedure in Example 1, except that 3,4-dichlorophenyl isocyanate was used instead of 3-chlorophenyl isocyanate. M.P.=124-129° C.; MS: (+)APCI (M+H)⁺ 328.; TLC: silica gel, Rf=0.4 (DCM-Hexane, 1:1, v/v. Elem. Anal. Calcd. for: C₁₅H₁₅C₁₂NO₃: C, 54.90%; H, 4.61%; N, 4.27%, Found: C, 53.67%; H, 4.06%; N, 4.57%; ¹H-NMR (DMSO-d₆) δ 8.00 (d, 1H); 7.60 (d, 1H); 7.50 (d-d,1H); 2.50 (d, 4H); 1.00 (s, 6H).

### Example 3

### N-(3,4-dichlorophenyl)-4,4-dimethyl-2,6-dioxocyclohexanecarbothioamide

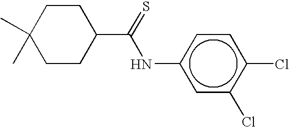

The title compound was prepared according to the procedure of Example 1, except that 3,4-dichloro-phenyl iso-thiocyanate was used instead of 3-chlorophenyl isocyanate. M.P:=113-116° C.; MS: EI M⁺ m/z 343; TLC: silica gel, Rf=0.72 (DCM-Hexane, 4:1, v/v); HPLC: C¹⁸-column, CH₃CN/H₂O, 75/25, v/v-0.1% H₃PO₃, 99.71%; Elem. Anal. Calcd. for C₁₅H₁₅C₁₂NO₂S: C, 52.33%; H, 4.39%; N, 4.07%, Found: C, 51.99%; H, 4.09%; N, 4.00%; ¹H-NMR (CDCl₃) δ 7.44 (s,1H); 7.3-7.0 (m, 2H); 2.43 and 2.30 (s, 4H); 0.90 (s, 6H).

### Example 4

### N-[4-chloro-3-(trifluoromethyl)phenyl]-4,4-dimethyl-2,6-dioxocyclohexane-carbothioamide

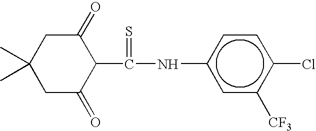

The title compound was prepared in a similar fashion to Example 3, using 4-chloro-3-trifluoro-phenyl isothiocyanate instead of 3,4-dichloro-phenyl iso-thiocyanate. M.P.=101-102° C.; MS, EI M⁺ m/z 377; TLC, silica gel, Rf=0.70 (DCM-Hexane, 4:1 v/v); Elem. Anal. Calcd. for C₁₆H₁₅CIF₃NO₂S, C, 50.87%; H, 4.00%; N, 3.71%, Found: C, 51.13%; H, 3.88%; N, 3.70%; ¹H-NMR (DMSO-d₆) δ 8.10 (s, 1H); 7.80 (q, 2H); 2.60 (s, 4H); 1.00 (s, 6H).

### Example 5

### N-[4-chloro-3-(trifluoromethyl)phenyl]-4,4-dimethyl-2,6-dioxocyclohexanecarboxamide

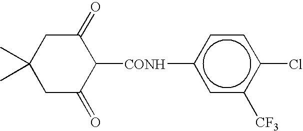

The title compound was prepared according to the procedure of Example 1, except that 3-trifluoromethyl-4-chloro-phenyl isocyanate was used instead of 3-chlorophenyl isocyanate. M.P:=128-131° C.; MS (−)APCI (M−H) 326; TLC, silica gel, Rf=0.35 (DCM-Hexane, 1:1, v/v); Elem. Anal. Calcd. for: C₁₆H₁₅CIF₃NO₃.0.3 H₂O: C, 52.27%; H, 4.29%; N, 3.81%, Found: C, 52.29%; H, 3.67%; N, 4.00%; ¹H-NMR (DMSO-d₆): δ 8.15 (s, 1H); 8-7.65 (m, 2H); 2.60 (s-broad, 4H); 1.00 (s, 6).

### Example 6

### 4,4-dimethyl-N-(2-nitrophenyl)-2,6-dioxocyclohexanecarboxamide

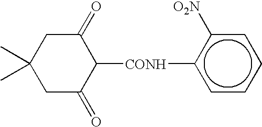

The title compound was prepared according to the procedure of Example 1, except that 2-nitro-phenyl isocyanate was used instead of 3-chlorophenyl isocyanate. MS: (+)APCI [M+H]⁺ 305; TLC: silica gel Rf=0.30 (DCM-Hexane, 1:1, v/v); Elem. Anal. Calcd. for C₁₅H₁₆N₂O₅: C, 59.21%; H, 5.30%, N, 9.21%, Found: C, 58.68%, H, 5.15%, N, 9.08; ¹H-NMR (DMSO-d₆) δ 8.20 (d, 1H); 8.10 (d, 1H); 7.75 (t, 1H); 7.40 (t, 1H); 2.65-2.40 (d, 4H); 1.00 (s, 6H).

### Example 7

### N-[4-chloro-2-(trifluoromethyl)phenyl]-4,4-dimethyl-2,6-dioxocyclohexanecarboxamide

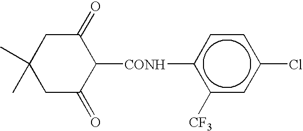

The title compound was prepared according to the procedure of Example 1 except that 2-trifluoromethyl-4-chloro-phenyl isocyanate was used instead of 3-chlorophenyl isocyanate.

MS: EI M⁺ 361, Elem. Anal. Calcd: For: C₁₆H₁₄CIF₃NO₃, C, 50.08%; H, 3.68%; N, 3.65%

Found: C, 49.42%; H, 3.45%; N, 3.49%.

### Example 8

### N-(3-trifluoromethylphenyl)-4,4-dimethyl-2,6-dioxocyclohexanecarboxamide

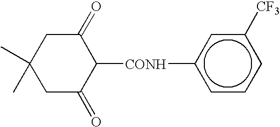

The title compound was prepared according to the procedure of Example 1, except that 3-trifluoromethylphenyl isocyanate was used instead of 3-chlorophenyl isocyanate. M.P:=116-117° C.; MS (−) ESI 326; TLC Silica gel. Rf=0.67 (DCM-Hexane, 4:1, v/v); Elem. Anal. Calcd. for C₁₆H₁₆F₃NO₃: C, 58.71%; H, 4.93%; N, 4.28%, Found: C, 58.69%; H, 4.81%; N, 4.23%; ¹H NMR (DMSO-d₆): δ 8.05 (s, 1H); 7.75 (d, 1H); 7.60 (t, 1H); 7.50 (d, 1H); 2.60-2.40 (d, 4H); 1.00 (s, 6H).

### Example 9

### N-[3,5-bis(trifluoromethyl)phenyl]-4,4-dimethyl-2,6-dioxocyclohexanecarboxamide

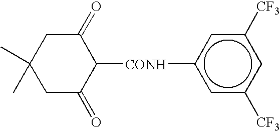

The title compound was prepared according to the procedure of Example 1, except that 3,5-bis(trifluoromethyl)phenyl isocyanate was used instead of 3-chlorophenyl isocyanate. MS (−)APCI 394; TLC Silica gel Rf=0.50 (DCM-hexane, 1:1, v/v); Elem. Anal. Calcd. for C₁₇H₁₅NF₆O₃: C, 51.65%; H, 3.82%; N, 3.54%, Found: C, 50.66%; H, 3.44%; N, 3.67%; ¹H-NMR (DMSO-d₆) δ 8.30 (s, 2H); 7.85 (s, 1H); 2.50 broad, 4H); 1.00 (s, 6H).

### Example 10

### N-(3,4-dichlorophenyl)-4-isopropyl-2,6-dioxocyclohexanecarboxamide

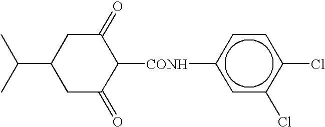

The title compound was prepared similar to the procedure in Example 1 where 5-isopropyl-cyclohexane-1,3-dione was used instead of 5,5-dimethylcyclohexane-1,3-dione, and 3,4-dichlorophenyl isocyanate was used instead of 3-chlorophenyl isocyanate. M.P. 138° C.; MS (+) APCI 342; TLC Silica gel Rf=0.50 (DCM-hexane, 3:2, v/v); Elem. Anal. Calcd. for: C₁₆H₁₇C₁₂NO₃: C, 56.16%; H, 5.01%; N, 4.09%, Found: C, 55.55%; H, 4.76%; N, 4.09%; ₁H-NMR (DMSO-d₆) δ 8.00 (s, 1H); 7.60 (d, 1H); 7.50 (d, 1H); 2.50 (s, 4H); 1.90 (m, 1H); 1.75 (m, 1H); 0.85 (d, 6H).

### Example 11

### N-[4-chloro-3-(trifluoromethyl)phenyl]-3,5-dioxotetrahydro-2H-thiopyran-4-carboxamide

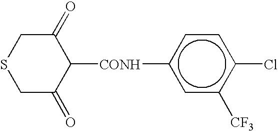

The title compound was prepared similar to the procedure in Example 1, where 3,5-dioxotetrahydrothiopyran was used instead of 5,5-dimethylcyclohexane-1,3-dione, and 3-trifluoromethyl-4-chlorophenyl isocyanate was used instead of 3-chlorophenyl isocyanate. M.P.=108-110° C.; MS (−)APCI 350; HPLC C¹⁸-column, CH₃CN—H₂O, 70/30-0.1% H₃PO₄, 99.99%; Elem. Anal. For C₁₃H₉CIF₃NO₃S: Calcd: C, 44.39%; H, 2.58%; N, 3.98%, Found: C, 44.36%; H, 2.50%; N 3.86%; ¹H-NMR (DMSO-d₆): δ 8.10 (d, 1H); 7.83 (q, 1H); 7.72 (d, 1H); 3.70 (s, 4H).

### Example 12

### 4,4-dimethyl-N-(4-nitrophenyl)-2,6-dioxocyclohexanecarboxamide

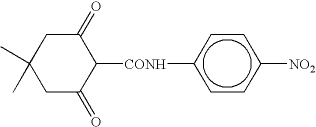

The title compound was prepared according to the procedure of Example 1, except that 4-nitro-phenyl isocyanate was used instead of 3-chlorophenyl isocyanate. TLC Silica gel. Rf=0.40 (DCM-Hexane, 4/1, v/v); Elem. Anal. For C₁₅H₁₆N₂O₅: Calcd:59.21%; H, 5.30%; N, 9.21%, Found: C, 58.88%; H, 5.23%; N, 9.15%; ¹H-NMR(DMSO-d₆) δ 8.25 (d, 2H); 7.85 (d, 2H); 2.65-2.40 (d, 4H); 1.00 (s,6H).

### Example 13

### N-(3-trifluoromethylphenyl)-4-ethyl-4-methyl-2,6-dioxocyclohexanecarboxamide

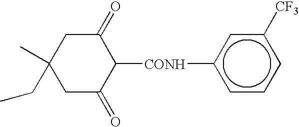

NaH (60% dispersion in mineral oil, 62 mg, 1.55 mmole) was added to a THF (20 ml) solution of 5-ethyl-3-hydroxy-5-methyl-cyclohex-2-enone (200 mg, 1.30 mmole), to which α,α,α-trifluoro-m-tolyl isocyanate (0.21 ml, 1.55 mmole) was added 15 minutes later. The reaction was stirred for 2 hours and quenched with water. The mixture was then acidified with 2 N HCl and extracted with ETOAc. The organic layer was collected, dried over MgSO₄ and concentrated. Column chromatography on silica gel eluted with 8% ETOAc in hexane gave the product as a white solid (385 mg). Yield 86.8%, M.P. 81-82° C., MS(−) ESI m/z=340.05[M−H]⁻. Elem. Anal. for C₁₇H₁₈F₃NO₃: Calcd: C, 59.82%; H, 5.32%; N, 4.10%, Found: C, 59.91%; H, 5.27%; N, 3.99%.

### Example 14

### N-(4-chloro-3-trifluoromethylphenyl)-4-ethyl-4-methyl-2,6-dioxocyclohexanecarboxamide

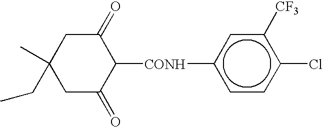

The title compound was prepared according to the procedure of Example 13 except that 4-chloro-3-(trifluoromethyl)phenyl isocyanate (343 mg, 1.55 mmole) was used instead of the α,α,α-trifluoro-m-tolyl isocyanate. A white solid was isolated (241 mg). Yield 49.3%, M.P. 87-88° C., MS(−) ESI m/z=374.03[M−H]⁻. Elem. Anal. for C₁₇H₁₇CIF₃NO₃: Calcd: C, 54.34%; H, 4.56%; N, 3.73%, Found: C, 54.35%; H, 4.52%; N, 3.60%.

### Example 15

### N-(3,4-dichlorophenyl)-4-ethyl-4-methyl-2,6-dioxocyclohexanecarboxamide

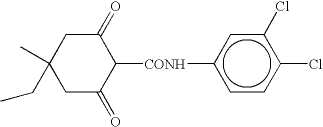

The title compound was prepared according to the procedure of Example 13 except that 3,4-dichlorophenyl isocyanate (292 mg, 1.55 mmole) was used instead of the α,α,α-trifluoro-m-tolyl isocyanate and the reaction was stirred overnight instead of 2 hours. A yellow solid was isolated (262 mg). Yield 76.6%, M.P. 74-75° C., MS(−) ESI m/z=3401.01[M−H]⁻. Elem. Anal. for C₁₆H₁₇Cl₂NO₃: Calcd: C, 56.16%; H, 5.01%; N, 4.09%, Found: C, 56.23%; H, 5.07%; N, 3.96%.

### Example 16

### N-(3-chloro-4-fluororphenyl)-4-ethyl-4-methyl-2,6-dioxocyclohexanecarboxamide

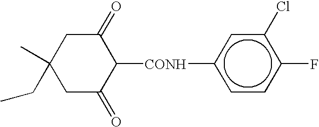

The title compound was prepared according to the procedure of Example 13 except that 3-chloro-4-fluorophenyl isocyanate (0.20 ml, 1.55 mmole) was used instead of the α,α,α-trifluoro-m-tolyl isocyanate and the reaction was stirred overnight instead of 2 hours. A yellow solid was isolated (331 mg). Yield 78.1%, M.P. 91-92° C., MS(−) ESI m/z=324.0[M−H]⁻. Elem. Anal. for C₁₆H₁₇FCINO₃: Calcd: C, 58.99%; H, 5.26%; N, 4.30%, Found: C, 58.93%; H, 5.05%; N, 4.15%.

### Example 17

### N-(4-trifluoromethylphenyl)-4-ethyl-4-methyl-2,6-dioxocyclohexanecarboxamide

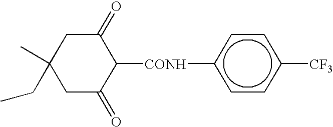

The title compound was prepared according to the procedure of Example 13 except that α,α,α-trifluoro-p-tolyl isocyanate (0.22 ml, 1.55 mmole) was used instead of the α,α,α-trifluoro-m-tolyl isocyanate and the reaction was stirred overnight instead of 2 hours. A yellow solid was isolated (176 mg). Yield 39.7%, M.P. 99-100° C., MS(−) ESI m/z=340.0[M−H]⁻. Elem. Anal. for C₁₇H₁₈F₃NO₃: Calcd: C, 59.82%; H, 5.32%; N, 4.10%, Found: C, 59.83%; H, 5.32%; N, 4.13%.

### Example 18

### N-(3,5-bis-trifluoromethylphenyl)-4-ethyl-4-methyl-2,6-dioxocyclohexanecarboxamide

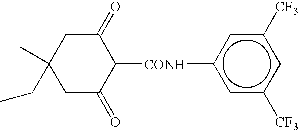

The title compound was prepared according to the procedure of Example 13 except that 3,5-bis-(trifluoromethyl)phenyl isocyanate (0.27 ml, 1.55 mmole) was used instead of the α,α,α-trifluoro-m-tolyl isocyanate. A yellow solid was isolated (321 mg). Yield 60.3%, M.P. 72-73° C., MS(−) ESI m/z=408.0[M−H]⁻. Elem. Anal. for C₁₈H₁₇F₆NO₃: Calcd: C, 52.82%; H, 4.19%; N, 3.42%, Found: C, 52.76%; H, 3.95%; N, 3.44%.

### Example 19

### N-[4-chloro-3-(trifluoromethyl)phenyl]-6,6-dimethyl-2,4-dioxotetrahydro-2H-pyran-3-carboxamide

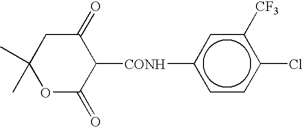

Triethylamine (0.78 ml, 5.6 mmole) was added into a DMF solution containing 6,6-dimethyl-dihydro-pyran-2,4-dione (500 mg, 3.52 mmole), to which 4-chloro-3-(trifluoromethyl)phenyl isocyanate (1.19 g, 5.28 mmole) was added after 10 minutes. The reaction was stirred for 3 hours. DMF and triethylamine were removed under reduced pressure, and the residue was dissolved in a mixture of EtOAc and water and acidified with HCl. The solution was extracted with EtOAc. The organic layer was collected, dried over MgSO₄ and concentrated to give the crude product, which was further crystallized to give the product as a white solid (141 mg). Yield 11.0%, M.P. 156.0-156.4° C., MS(−) ESI m/z=362[M−H]⁻. Elem. Anal. for C₁₅H₁₃CIF₃NO₄: Calcd: C, 49.53%; H, 3.60%; N, 3.85%, Found: C, 49.39%; H, 3.34%; N, 4.04%.

### Example 20

### N-[4-(trifluoromethyl)phenyl]-6,6-dimethyl-2,4-dioxotetrahydro-2H-pyran-3-carboxamide

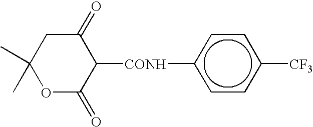

Triethylamine (0.40 ml, 2.87 mmole) was added into a DMF solution containing 6,6-dimethyl-dihydro-pyran-2,4-dione (260 mg, 1.83 mmole), to which 4-(trifluoromethyl)phenyl isocyanate (449.1 mg, 2.40 mmole) was added after 10 minutes. The reaction was stirred for 3 hours. DMF and triethylamine were removed under reduced pressure, and the residue was dissolved in a mixture of EtOAc and water and acidified with HCl. The solution was extracted with EtOAc. The organic layer was collected, dried over MgSO₄ and concentrated to give the crude product, which was further crystallized to give the product as a white solid (295 mg). Yield 89.6%, M.P. 175.9-176.4° C., MS(−) ESI m/z=328[M−H]⁻. Elem. Anal. for C₁₅H₁₄F₃NO₄: Calcd: C, 54.72%; H, 4.29%; N, 4.25%, Found: C, 54.72%; H, 4.10%; N, 4.13%.

### Example 21

### N-[3-(trifluoromethyl)phenyl]-6,6-dimethyl-2,4-dioxotetrahydro-2H-pyran-3-carboxamide

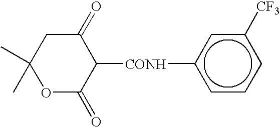

Triethylamine (0.38 ml, 2.75 mmole) was added into a DMF solution containing 6,6-dimethyl-dihydro-pyran-2,4-dione (260 mg, 1.83 mmole), to which 3-(trifluoromethyl)phenyl isocyanate (530 mg, 2.75 mmole) was added after 10 minutes. The reaction was stirred for 3 hours. DMF and triethylamine were removed under reduced pressure, and the residue was dissolved in a mixture of EtOAc and water. The solution was extracted with EtOAc. The organic layer was collected, dried over MgSO₄ and concentrated to give the crude product, which was further crystallized to give the product as a white solid (91 mg). Yield 10.1%, M.P. 149.6-150.1° C., MS(−) ESI m/z=328[M−H]⁻. Elem. Anal. for C₁₅H₁₄F₃NO₄: Calcd: C, 54.72%; H, 4.29%; N, 4.25%, Found: C, 53.44%; H, 4.19%; N, 4.06%.

### Example 22

### N-(3-nitrophenyl)-6,6-dimethyl-2,4-dioxotetrahydro-2H-pyran-3-carboxamide

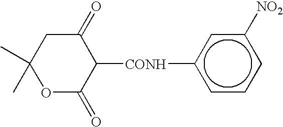

Triethylamine (0.44 ml, 3.17 mmole) was added into a DMF solution containing 6,6-dimethyl-dihydro-pyran-2,4-dione (300 mg, 2.11 mmole), to which 3-nitrophenyl isocyanate (357 mg, 2.11 mmole) was added after 10 minutes. The reaction was stirred for 3 hours. DMF and triethylamine were removed under reduced pressure, and the residue was dissolved in a mixture of EtOAc and water. The solution was extracted with EtOAc. The organic layer was collected, dried over MgSO₄ and concentrated to give the crude product, which was further purified by column chromatography eluted with CHCl₃ to give the product as an orange solid (210 mg).

Yield 32.5%, M.P. 138.4-139.6° C., MS(−) ESI m/z=305[M−H]⁻. Elem. Anal. for C₁₄H₁₄N₂O₆: Calcd: C, 54.90%; H, 4.61%; N, 9.15%, Found: C, 54.84%; H, 4.48%; N, 9.23%.

### Example 23

### N-(3,4-dichlorophenyl)-6,6-dimethyl-2,4-dioxotetrahydro-2H-pyran-3-carboxamide

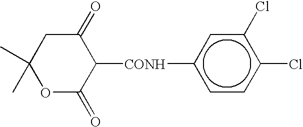

Triethylamine (0.78 ml, 5.62 mmole) was added into a DMF solution containing 6,6-dimethyl-dihydro-pyran-2,4-dione (400 mg, 2.81 mmole), to which 3,4-dichlorophenyl isocyanate (540 mg, 2.87 mmole) was added after 10 minutes. The reaction was stirred for 4 hours. DMF and triethylamine were removed under reduced pressure, and the residue was dissolved in a mixture of EtOAc and water. The solution was extracted with EtOAc. The organic layer was collected, dried over MgSO₄ and concentrated to give the crude product, which was further purified by column chromatography to give the product as a white solid (224 mg). Yield 24.2%, M.P. 212-215° C., MS(−) ESI m/z=328[M−H]⁻. Elem. Anal. for C₁₄H₁₃C₁₂NO₄: Calcd: C, 50.93%; H, 3.97%; N, 4.24%, Found: C, 48.81%; H, 3.78%; N, 3.77%.

### Example 24

### N-[3-(trifluoromethyl)phenyl]-5,5-dimethyl-2,4-dioxotetrahydro-2H-pyran-3-carboxamide

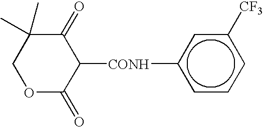

5,5-Dimethyl-dihydro-pyran-2,4-dione (225 mg, 1.6 mmole) was added into a DMF solution containing NaH (60% dispersion in mineral oil, 76.8 mg, 1.92 mmole), to which α,α,α-trifluoro-m-tolylisocyanate (0.22 ml, 1.6 mmole) was added after 15 minutes. The reaction was stirred for 3 hours and then quenched with water. The mixture was acidified with diluted H₂SO₄ and extracted with EtOAc. The organic layer was collected, washed with saturated aqueous NaCl solution, dried over MgSO₄ and concentrated to give the crude product. Further purification by HPLC yielded the product as a white solid (213 mg). Yield 40.4%, M.P. above 200° C., MS(−) ESI m/z=328[M−H]⁻. Elem. Anal. for C₁₅H₁₄F₃NO₄: Calcd: C, 54.72%; H, 4.29%; N, 4.25%, Found: C, 51.09%; H, 3.86%; N, 4.06%.

### Example 25

### N-[4-(trifluoromethyl)phenyl]-5,5-dimethyl-2,4-dioxotetrahydro-2H-pyran-3-carboxamide

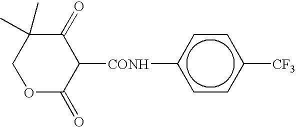

The title compound was prepared according to the procedure of Example 24 except that α,α,α-trifluoro-p-tolylisocyanate (0.23 ml, 1.6 mmole) was used instead of the α,α,α-trifluoro-m-tolylisocyanate. A white solid (311 mg) was isolated. Yield 59.0%, M.P. above 200° C., MS(−) ESI m/z=328[M−H]⁻. Elem. Anal. for C₁₅H₁₄F₃NO₄: Calcd: C, 54.72%; H, 4.29%; N, 4.25%, Found: C, 52.59%; H, 3.74%; N, 4.66%.

### Example 26

### N-[4-chloro-3-(trifluoromethyl)phenyl]-5,5-dimethyl-2,4-dioxotetrahydro-2H-pyran-3-carboxamide

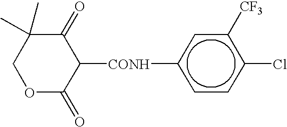

The title compound was prepared according to the procedure of Example 24 except that 4-chloro-3-(trifluoromethyl)phenylisocyanate (354.5 mg, 1.6 mmole) was used instead of the α,α,α-trifluoro-m-tolylisocyanate. A white solid (421 mg) was isolated.

Yield 72.5%, M.P. 144-145° C., MS(−) ESI m/z=364[M−H]⁻. Elem. Anal. for C₁₅H₁₃CIF₃NO₄: Calcd: C, 49.53%; H, 3.60%; N, 3.85%, Found: C, 49.88%; H, 3.28%; N, 4.14%.

### Example 27

### 3-Fluoro-4,4-dimethyl-2,6-dioxo-cyclohexanecarboxylic acid(4-trifluoromethyl-phenyl)-amide

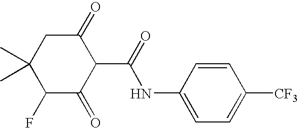

0.6 g (0.0037 mole) of 4-fluoro-5,5-dimethyl-cyclohexane-1,3-dione was stirred in 40 ml acetone. 0.5 ml triethylamine was added and the reaction was stirred for 5 minutes. Then, 0.52 ml of α,α,α-trifluoro-p-tolyl isocyanate was added. The reaction was stirred overnight at room temperature. The acetone was stripped via a rotoevaporator and the residue was taken up in 50 ml of ethyl acetate. The organic layer was washed with 2 N HCl and brine. The organic layer was dried over sodium sulfate, filtered and stripped to a yellow solid (crude 1.2 g). The crude product was taken up in 1/1 hexane/methylene chloride (8 ml) and filtered. The filtrate was charged on a Biotage Flash 90 cartridge (silica). The product was eluted with 1/1 hexane/methylene chloride. The weight of the pure product was 0.34 g (26% yield).

MP=129° C., MS: (−) ES (M−H)⁻ 344.07, Elemental Anal. Calcd. for C₁₆H₁₅F₄NO₃: C, 55.66%, H, 4.38%; N, 4.06%, Found: C, 55.60%; H, 4.31%; N,4.00%. ¹—H NMR (DMSO-d₆): δ 16.97 (b, 1H); 11.45 (b, 1H); 8.08 (s, 1H); 7.81( d, 1H); 7.63 (t, 1H); 7.56 (d, 1H); 5.16,5.06; (b, 1H); 2.92 (b, 1H); 2.61 (b, 1H); 1.17 (s, 3H); 0.99 (s, 3H).

### Example 28

### 3-Fluoro-4,4-dimethyl-2,6-dioxo-cyclohexanecarboxylic acid(3,5-dichloro-phenyl)-amide

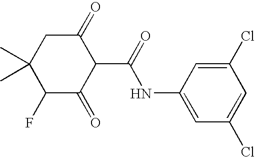

The title compound was prepared similar to the procedure of Example 27, where 3,5-dichlorophenyl isocyanate was used instead of α,α,α-trifluoro-p-tolyl isocyanate.

MS: (+) ES (M+H)⁺ 344/346 (2CL), Elemental Anal. Calcd. for C₁₅H₁₄C₁₂FNO₃+1.0 H₂O: C, 49.47%; H,4.43%; N,3.85%, Found: C, 49.91%; H, 3.66%; N, 3.88%,

¹H-NMR (DMSO-d₆): δ 11.35 (b, 1H); 7.72 (s, 2H); 7.42 (s, 1H) ; 6.52 (s, 1H); 5.15 (b, 1H); 5.06 (b, 1H); 2.92 (d, 1H); 2.64 (d, 1H); 1.16 ( s, 3H) 0.98 (s, 3H).

### Example 29

### 3-Fluoro-4,4-dimethyl-2,6-dioxo-cyclohexanecarboxylic acid(3,5-difluoro-phenyl)-amide

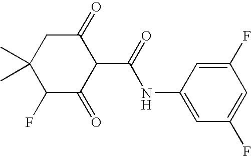

The title compound was prepared similar to the procedure of Example 27, where 3,5-difluorophenyl isocyanate was used instead of α,α,α-trifluoro-p-tolyl isocyanate.

MP=131-132° C.,

MS: (−) ES (M−H)⁻ 312/313, Elemental Anal. Calcd. for C₁₅H₁₄F₃NO₃: C, 57.51%; H,4.50%; N,4.47%, Found: C, 57.30%; H, 4.46%; N, 4.36%, ¹H-NMR (DMSO-d₆): δ 16.71 (b, 1H); 11.41 (b, 1H); 7.42 (d,2H); 7.09 (t, 1H); 5.15 5.06 (b, 1H); 2.95 (d, 1H); 2.63 (m, 1H); 1.16 (s, 3H); 0.98 (s, 3H).

### Example 30

### 4,4-Dimethyl-2,6-dioxo-3-phenyl-cyclohexanecarboxylic acid(3-trifluoromethyl-phenyl)-amide

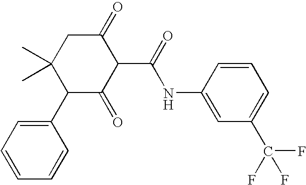

0.115 g (0.00262 mole) of 60% oil dispersed sodium hydride was stirred in 40 ml tetrahyrofuran (THF). 0.57 g (0.00262 mole of 5,5 dimethyl-4-phenyl-cyclohexane-1,3-dione was added. The reaction was stirred for fifteen minutes before adding 0.36 ml (0.00262 mole) of α,α,α-trifluoro-m-tolyl isocynate. The reaction was refluxed overnight. The tetrahydrofuran (THF) was removed via a rotoevaporator. The residue was taken up in ethyl acetate and washed twice with 2N HCl and once with brine. The ethyl acetate layer was dried over sodium sulfate, filtered and stripped to a yellow oil (1.1 g). The crude product was taken up in 10 ml of 4/1 hexane/ethyl acetate and charged on a Biotage Flash 90 cartridge. The pure product (0.7 g-67% yield) was eluted with 4/1 hexane/ethyl acetate.

MS: (+) ES (M+H)⁺ 404, Elemental Anal. Calcd. For C₂₂H₂₀F₃NO₃: C, 65.50%; H, 5.00%; N, 3.47%, Found: c, 65.30%; H, 4.96%; N, 3.37%, ¹H-NMR (DMSO-d₆): δ 17.22 (s, 1H); 12.02 (bd,. 1H): 8.09 (s, 1H); 7.80 (d, 1H); 7.61 (s. 1H); 7.54 (s, 1H); 7.34 (m, 5H); 3.95-3.78 (bd, 2H); 2.95-2.66 (bd, 2H); 1.16 (m, 2H); 0.86 (m, 3H).

### Example 31

### 2,4-Dioxo-3-(3-trifluoromethyl-phenylcarbamoyl)-decahydro-naphthalene-1-carboxylic acid ethyl ester

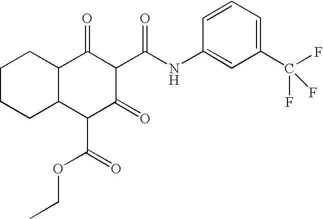

(0.13 g 0.0033 mole) of 60% oil dispersed sodium hydride was stirred in 20 ml THF. 0.7 g (0.003 mole) of 2,4-dioxodecahydro-napthalene-1-carboxylic acid ethyl ester in 60 ml THF was added. 0.4 ml (0.003 mole) of α,α,α-trifluoro-m-tolyl isocyanate was added. The reaction was stirred overnight at room temperature. The solvent was removed via rotoevaporator/water aspirator. The residue was taken up in ethyl acetate and was washed twice with 2N HCl, and once with brine. The organic layer was dried over Na₂SO₄ filtered and stripped to a yellow oil (1.2 gm—crude). The crude oil was taken up in 10 ml 4/1 hexane/ethyl acetate and charged on a Biotage Flash 90 cartridge (silica). 0.5 (40% yield) gm of the title compound was isolated as colorless oil.

MS: (−)ES [M−H] 424.13, Elem. Anal. Calcd. for C₂₁H₂₂F₃NO₅: C, 59.29%; H, 5.21% N, 3.14%, Found: C, 59.10%; H, 5.23% N, 3.14%, ¹H-NMR (DMSO-d₆) δ 17.39 (b, 1H), 11.70 (0.1H), 8.05 (d, 1H), 7.81 (d, 1H), 7.64 (+, 1H), 7.54 (d, 1H), 4.19 (m, 2H), 3.91 (d, 1H), 2.79 (b, 1H), 2.59 (b, 1H), 1.97-1.2 (b, 7H), 1.17 (m, 3H)

HPLC C¹⁸ 70/30 CAN/H₂O, mixture of diastereomers (74/21).

### Example 32

### 3-Methyl-2,6-dioxo-3-phenyl-cyclohexanecarboxylic acid(3-trifluoromethyl-phenyl)-amide

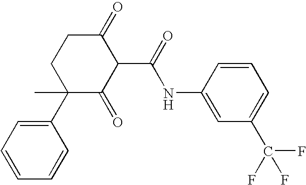

4-methyl-4-phenyl-cyclohexane-1,3-dione (0.45 g 0.0022 mole) was stirred in 20 ml dimethylformamide (DMF). 0.097 g (0.0022 mole) of 60% oil dispersed sodium hydride was added and the reaction was stirred for twenty minutes. 0.3 ml (0.0022 mole of α,α,α-trifluoro-m-tolyl-isocyanate was added and the reaction was stirred overnight. The solvent was removed via a vacuum pump/rotoevaporator. The residue was taken up in ethyl acetate and washed twice with 1N HCl and once with brine. The ethyl acetate layer was dried over sodium sulfate, filtered and stripped to a yellow oil (0.82 g). The crude product was taken up in methylene chloride and charged on Biotage Flash 90 cartridge(silica). Pure product (0.28 g 33% yield) was eluted with methylene chloride.

MS: (−) ES (M−H)⁻ 388, Elemental Anal. Calcd. for C₂₁H₁₈F₃NO₃: C, 64.78%; H, 4.66%; 3.60%

Found: C, 65.40%; H, 4.93%; N, 3.31%, ¹H-NMR (DMSO-d₆): δ 17.10 (b, 1H); 12.15 (b, 1H); 8.10 (s, 1H); 7.93 (s, 1H); 7.64 (m, 1H); 7.53 (m, 1H); 7.34 (m ,5H); 2.75-2.05 (bm, 4H); 1.4 (s, 3H).

### Example 33

### 3-Methoxymethyl-4,4-dimethyl-2,6-dioxo-cyclohexanecarboxylic acid (3-trifluoromethyl-phenyl)-amide

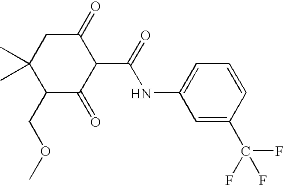

0.8 g (0.0043 mole) of 4-methoxymethyl-5,5-dimethylcyclohexane-1,3-dione was stirred in 30 ml of tetrahydrofuran with 0.189 g (0.0043 mole) of 60% oil dispersed sodium hydride for thirty minutes. α,α,α trifluoro-m-tolyl isocyanate (0.9 ml-0.0064 mole) was added and the reaction was stirred overnight. The solvent was removed via a rotoevaporator. The residue was taken up in ethyl acetate and washed twice with 2N HCl. The ethyl acetate layer was dried over sodium sulfate, filtered, and stripped to a brown oil (1.3 g). The crude product was taken up in 10 ml of methylene chloride and charged on a Biotage Flash 90 cartridge (silica). 0.098 g of pure product was eluted with 1/1 methylene/hexane.

MS: (−) ES (M−H)⁻ 370, ¹H-NMR (CDCl₃) δ 17.42(s, 1H); 12.00 (s, 1H); 7.96 (s, 1H); 7.78 (m,1H); 7.48 (m, 1H); 7.43 (d, 1H); 3.80 (m, 2H); 3.34 (d, 3H); 2.90 (t, 1H); 2.45 (m, 2H); 1.12 (m, 6H).

### Example 34

### 7,9-Dioxo-6,10-dioxa-spiro[4.5]decane-8-carbothioic acid(4-chloro-3-trifluoromethyl-phenyl)-amide

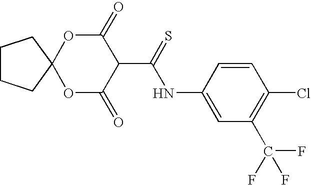

0.72 gms (0.0042 mole) of 6,10-dioxa-spiro[4.5]decane-7,9-dione were stirred in 20 mls. of dry dimethylformamide (DMF) with 0.168 gm. (0.0042 mole) of 60% oil dispersed sodium hydride for 30 minutes. 1.0 gm. (0.0042 mole) of 4-chloro-3-(trifluoromethyl)phenylisothiocyanate was added and the reaction was stirred overnight at room temperature. The DMF was removed via vacuum pump. Residue was taken up in ethyl acetate and washed with 0.5N HCl followed by brine. Organic layer was dried over sodium sulfate, filtered and stripped to a brown oil (1.5 gm). The crude product was charged on a Biotage flash 90 cartridge (silica) and eluted with 4/1 methylene chloride/hexane. 0.194 gms. of compound (gray crystals) was isolated (11% yield).

MS: (+) EI (M+H)⁺ 407/409, Elemental Anal. Calcd. for C₁₆H₁₃CLF₃NO₄S+0.75 H₂O: C, 45,61%; H,3.47%; N, 3.32%, Found: C,45.61%; H, 3.02%; N, 3.28%, ¹H-NMR (DMSO-d₆) δ 12.9 (s, 12.95) (s, 1H); 8.50 (d, 1H);7.85 (d,1H); 1.98(m, 4H); 1.69 (m, 4H).

### Example 35

### Evaluation of Compounds for Inhibiting the Inactivation of Kv1.1/Kvβ1 Potassium ion Channel Complex

Compounds useful in this invention were examined for their ability to inhibit the inactivation of the human Kv1.1/Kvβ1 potassium ion channel complex in vitro using electrophysiological current recordings on inactivating Kv1.1/Kvβ1channels expressed in Xenopus oocytes. The following procedure was used.

Xenopus oocytes were harvested from frogs under general anesthesia using aseptic techniques. Oocytes were treated with 2 mg/ml collagenase for 1-1.5 hr, defolliculated, and injected with with 40 nL cRNA (a mixture of hKv1.1 and hKvβ1) produced using standard molecular biological techniques (Sambrook, et al., 1989, Molecular Cloning: A Laboratory Manual). The concentration of hKv1.1/hKvβ1 cRNA varies, but was generally about 50 ng/μL hKv1.1 and 0.5-1.5 mg/μL hkβ1.

Currents were recorded using standard voltage clamp amplifiers at room temperature. Recordings were performed in the bath solution “ND-96” containing 96 mM NaCl, 2 mM KCl, 1 mm CaCl₂, 1 mM MgCl₂, 10 mM HEPES and 50 mg/μL Gentamycin (pH=7.6). The pipette (electrode) solution generally consisted of 3 mM KCl. Electrodes were made from WPI TW150F-4 borosilicate glass (or equivalent) and typically had resistances of 0.5-1.5 Mohms. The standard current—voltage (IV) pulse protocol consisted of voltage steps from −60 mV to +50 mV, in 10 mV increments for 200 ms, at a frequency of one IV set per 2 minutes. Data were digitized and analyzed by standard software packages.

Test compounds where first dissolved in DMSO to an initial concentration of 1-100 mM. Working dilutions of 1:2000 to 1:500 were made in the bath solution for final concentrations in the micromolar range. The bath solutions containing the test compounds were applied by bath perfusion using gravity or pump-driven flow. A minimum of 2 mL (10 times the bath volume) over 3 minutes were flowed for each compound concentration tested. Data were averaged across at least 3 cells tested for each concentration.

Test compounds were examined for their ability to inhibit the inactivation of the steady state portion of the IV curve. Results are presented as a percent increase in the steady state current relative to control experiments in which oocytes were treated with bath solution which did not contain test compound. The results are shown in Table 1 below. As the results show, compounds of this displayed the ability to inhibit the inactivation of the steady state current of the human Kv1.1/Kvβ1 potassium ion channel complex, as measured by the increase in steady state current versus control.

TABLE 1
Results of Compounds for Inhibiting Inactivation
of Kv1.1/Kvβ1 potassium ion channel complex

| | |
|---|---|
| | Percent Increase in Steady State Current |
| Example | (@ concentration shown in parentheses) |

| | | | |
|---|---|---|---|
| 1 | 21% | (100 | μm) |
| 2 | 75% | (100 | μm) |
| 3 | — | (300 | μm) |
| 4 | 9% | (300 | μm) |
| 5 | 16% | (50 | μm) |
| 6 | 58% | (100 | μm) |
| 7 | 45% | (100 | μm) |
| 8 | 40% | (100 | μm) |
| 9 | 20% | (50 | μm) |
| 10 | 38% | (50 | μm) |
| 11 | 106% | (50 | μm) |
| 12 | 104% | (100 | μm) |
| 13 | 9% | (50 | μm) |
| 14 | 98% | (50 | μm) |
| 15 | 64% | (50 | μm) |
| 16 | 18% | (50 | μm) |
| 17 | 105% | (50 | μm) |
| 18 | 106% | (50 | μm) |
| 19 | 68% | (50 | μm) |
| 20 | 17% | (50 | μm) |
| 21 | 13% | (50 | μm) |
| 22 | 10% | (50 | μm) |
| 23 | 22% | (50 | μm) |
| 24 | 5% | (50 | μm) |
| 25 | 10% | (50 | μm) |
| 26 | 173% | (50 | μm) |
| 27 | 191% | (50 | μm) |
| 28 | 279% | (50 | μm) |
| 29 | 258% | (50 | μm) |
| 30 | 8% | (50 | μm) |
| 31 | 227% | (50 | μm) |
| 32 | 6% | (50 | μm) |
| 33 | 1% | (50 | μm) |
| 34 | 95% | (300 | μm) |

### Example 36

### Evaluation of Compounds for Inhibiting PTZ Induced Seizures

Compounds useful in this invention were also examined for their anticonvulsant activity in vivo by assessing their ability to inhibit the seizures induced by administration of pentylenetetrazole (PTZ). Adult mice were pretreated intraperitoneally with the test compound. Thirty minutes later these animals were challenged with pentylenetetrazole (85 mg/kg, s.c.) and observed for onset of seizures during a 30 minute test period. Compounds of this invention inhibited the convulsions induced by treatment with PTZ. ED₅₀ values for the compounds of this invention varied, but generally fell within the dose range of 10-100 mg/kg i.p. The results are report in Table 2 below.

TABLE 2
Results of Compounds for Inhibiting PTZ Induced Seizures

| Example | Dose Range (mg/kg) |
|---|---|
| 1 | ED₅₀ = 30 |
| 2 | ED₅₀ = 50 |
| 9 | ED₅₀ = 55 |
| 8 | ED₅₀ = 32 |

### Example 37

### Evaluation of Compounds for Anxiolytic Activity

Anxiolytic activity of the compounds of this invention was determined using the mouse 4-Plate model, originally described by Boissier et al. (Eur. J. Pharmacol. (1968) 4:145-151). The test procedure used is described in further detail below. Anxiolytic efficacy is measured as the ability of the test compound to increase the number of times that an animal will accept a mild punishment (in the form of a foot shock) in order to explore its environment. A number of clinically effective treatments for anxiety from multiple pharmacological classes are effective in this model (Hascoet et al., Pharmacol. Biochem. Beh. (2000) 65:339-344).

The compound of Example 8 was examined in the 4-Plate model. As can be seen from the data in Table 3, the compound of Example 8, when administered at a dose of 56 mg/kg ip, increased the number of punished crossings by 31%. Alprazolam, a known anxiolytic benzodiazepine derivative, also displayed efficacy in this model, increasing the number of punished crossings by 70% at therapeutically relevant doses.

TABLE 3
Results of Compounds for Anxiolytic Activity

| | % Time Spent in Open Zones |
|---|---|
| Compound/Dose | (Relative to Control) |
| Alprazolam (0.3 mg/kg ip.) | 170% |
| Example 8 (60 mg/kg ip.) | 131% |

Experimental Protocol for the 4-Plate Model

The “four-plate” apparatus consisted of a Plexiglas cage (18×25×16 cm) floored by four identical rectangular metal plates (8×11 cm) separated from one another by a gap of 4 mm. The plates were connected to a device that generated electric shocks (0.8 mA, 0.5 sec). This intensity of shock was demonstrated to reduce punished crossings in controls. Test animals (male Swiss Webster mice) were group housed with ad-lib access to food and water. Compounds were administered at the appropriate dose ip. 30 min prior to testing. Control animals were given the vehicle without drug. Animals were then placed in the 4-plate apparatus and allowed to habituate for 18 seconds. Following the 18-sec habituation period, an electric shock (0.8 mA, 0.5 sec) was delivered to the mouse when crossing from one plate to another. No shocks were delivered during the 3 seconds following shock administration. The number of punished crossings was recorded during a 1-min testing period. Data were expressed as the percent change in the number of punished crossings relative to control.

What is claimed is:

1. A method for treating one or more conditions associated with the abnormal inactivation of one or more Kv1.1 voltage-gated potassium channels comprising administering to a mammal a pharmaceutically effective amount of at least one compound of formula I, or a tautomer, or a pharmaceutically acceptable salt thereof or both: 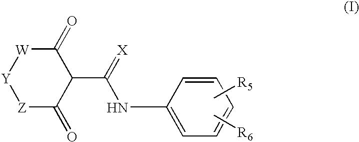
wherein:
Y is CR₁R₂, S or O;
W is CR₉R₁₀ or O;
Z is CR₃R₄ or O; with the proviso that when Y is O or S, W is CR₉R₁₀ and Z is CR₃R₄, and with the proviso that when W or Z or both are O, Y is CR₁R₂;
X is O or S;
R₁, R₂, R₃, R₄, R₉, and R₁₀ are independently selected from hydrogen, a halogen atom, a hydroxyl group, a C₁ to C₆ alkyl group, a C₁ to C₆ alkoxy group, a C₁ to C₁₂ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, or alkanesulfonyl; or two of R₁, R₂, R₃, R₄, R₉, or R₁₀, attached to the same carbon atom or adjacent carbon atoms, may form together with the carbon atom or carbon atoms, in spiro or fused form, a carbocyclic or heterocyclic ring having 3 to 8 carbon atoms and zero to two heteroatoms independently selected from N, O, or S;
R₅ and R₆ are independently selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl; and
R₇ and R₈ are independently selected from hydrogen or a C₁ to C₆ alkyl group;
wherein any moiety containing an alkyl, alkenyl, carbocyclic, heterocyclic, cycloalkyl, heterocycloalkyl, aryl, or heteroaryl group may optionally be substituted with one to three substituents independently selected from a halogen atom, CN, NO₂ or hydroxyl group, a C₁-C₆ alkyl group, a C₁-C₆ alkoxy group, a C₁-C₄ perhalogenated alkyl group or C₁-C₄ perhalogenated alkoxy group.

2. The method of claim 1 wherein at least one compound administered has the formula I, or a tautomer thereof, or a pharmaceutically acceptable salt thereof or both wherein:
Y is CR₁R₂;
W is CR₉R₁₀;
Z is CR₃R₄; and
R₁, R₂, R₃, R₄, R₉ and R₁₀ are independently selected from hydrogen, a halogen atom, a hydroxyl group, a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group, a C₂ to C₇ alkenyl group, C₅ to C₇ aryl group, C₆ to C₁₃ arylalkyl group, a C₅ to C₇ aryloxy group, C₆ to C₁₃ arylalkyloxy group, CN, NO₂, NR₇R₈, CO₂R₇, COR₇, or alkanesulfonyl.

3. The method of claim 2 wherein one of i) R₁ and R₂, ii) R₃ and R₄ or iii) R₉ and R₁₀ are each independently selected from a C₁ to C₄ alkyl group, X is O, and at least one of R₅ or R₆ is a halogen atom, a C₁ to C₄ perhalogenated alkyl group, a C₁ to C₄ perhalogenated alkoxy group, or NO₂.

4. The method of claim 2 wherein the compound administered is selected from at least one of:
N-(3,4-dichlorophenyl)-4-(isopropyl)-2,6-dioxocyclohexanecarboxamide;
N-(3-chlorophenyl)-4-methyl-2,6-dioxocyclohexanecarboxamide;
N-(3,4-dichlorophenyl)-4,4-dimethyl-2,6-dioxo-cyclohexanecarboxamide;
N-(3-trifluoromethylphenyl)-4,4-dimethyl-2,6-dioxocyclohexanecarboxamide;
N-(3,5-bis-trifluoromethylphenyl)-4,4-dimethyl-2,6-dioxocyclohexanecarboxamide;
N-(4-trifluoromethylphenyl)-4,4-dimethyl-2,6-dioxocyclohexanecarboxamide;
N-(4-fluorophenyl)-4,4-dimethyl-2,6-dioxocyclohexanecarboxamide;
N-(3-trifluoromethoxyphenyl)-4,4-dimethyl-2,6-dioxocyclohexanecarboxamide;
N-(3,4-difluorophenyl)-4,4-dimethyl-2,6-dioxo-cyclohexanecarboxamide;
N-[4-chloro-2-(trifluoromethyl)phenyl]-4,4-dimethyl-2,6-dioxocyclohexanecarboxamide;
4,4-dimethyl-N-(2-nitrophenyl)-2,6-dioxocyclohexanecarboxamide;
4,4-dimethyl-N-(4-nitrophenyl)-2,6-dioxocyclohexanecarboxamide;
N-[4-chloro-3-(trifluoromethyl)phenyl]-4,4-dimethyl-2,6-dioxocyclohexanecarboxamide;
N-(3-chlorophenyl)-4,4-dimethyl-2,6-dioxocyclohexanecarboxamide;
N-(3,4-dichlorophenyl)-4,4-dimethyl-2,6-dioxocyclohexanecarbothioamide;
N-[4-chloro-3-(trifluoromethyl)phenyl]-4,4-dimethyl-2,6-dioxocyclohexanecarbothioamide;
N-(3-chloro-phenyl)-3,3-dimethyl-2,6-dioxocyclohexanecarboxamide;
N-(3,4-dichlorophenyl)-3,3-dimethyl-2,6-dioxocyclohexanecarboxamide;
N-(3-trifluoromethylphenyl)-3,3-dimethyl-2,6-dioxocyclohexanecarboxamide;
N-(3,5-bis-trifluoromethylphenyl)-3,3-dimethyl-2,6-dioxocyclohexanecarboxamide;
N-(4-trifluoromethylphenyl)-3,3-dimethyl-2,6-dioxocyclohexanecarboxamide;
N-(4-fluorophenyl)-3,3-dimethyl-2,6-dioxocyclohexanecarboxamide;
N-(3-trifluoromethoxyphenyl)-3,3-dimethyl-2,6-dioxocyclohexanecarboxamide;
N-(3,4-difluorophenyl)-3,3-dimethyl-2,6-dioxocyclohexanecarboxamide;
N-(3-trifluoromethylphenyl)-4-ethyl-4-methyl-2,6-dioxocyclohexanecarboxamide;
N-(4-chloro-3-trifluoromethylphenyl)-4-ethyl-4-methyl-2,6-dioxocyclohexanecarboxamide;
N-(3,4-dichlorophenyl)-4-ethyl-4-methyl-2,6-dioxocyclohexanecarboxamide;
N-(3-chloro-4-fluorophenyl)-4-ethyl-4-methyl-2,6-dioxocyclohexanecarboxamide;
N-(4-trifluoromethylphenyl)-4-ethyl-4-methyl-2,6-dioxocyclohexanecarboxamide;
N-(3,5-bis-trifluoromethylphenyl)-4-ethyl-4-methyl-2,6-dioxocyclohexanecarboxamide;
3-Fluoro-4,4-dimethyl-2,6-dioxo-cyclohexanecarboxylic acid(4-trifluoromethyl-phenyl)-amide;
3-Fluoro-4,4-dimethyl-2,6-dioxo-cyclohexanecarboxylic acid(3,5-dichloro-phenyl)-amide;
3-Fluoro-4,4-dimethyl-2,6-dioxo-cyclohexanecarboxylic acid(3,5-difluoro-phenyl)-amide;
4,4-Dimethyl-2,6-dioxo-3-phenyl-cyclohexanecarboxylic acid(3-trifluoromethyl-phenyl)-amide;
3-Methyl-2,6-dioxo-3-phenyl-cyclohexanecarboxylic acid(3-trifluoromethyl-phenyl)-amide;
3-Methoxymethyl-4,4-dimethyl-2,6-dioxo-cyclohexanecarboxylic acid(3-trifluoromethyl-phenyl)-amide; or
a tautomer thereof, or a pharmaceutically acceptable salt thereof or both.

5. The method of claim 2 wherein the compound administered is selected from at least one of:
N-(3,4-dichlorophenyl)-4-(isopropyl)-2,6-dioxocyclohexanecarboxamide;
N-(3-chlorophenyl)-4-methyl-2,6-dioxocyclohexanecarboxamide;
N-(3,4-dichlorophenyl)-4,4-dimethyl-2,6-dioxo-cyclohexanecarboxamide;
N-(3-trifluoromethylphenyl)-4,4-dimethyl-2,6-dioxocyclohexanecarboxamide;
N-(3,5-bis-trifluoromethylphenyl)-4,4-dimethyl-2,6-dioxocyclohexanecarboxamide;
N-(4-trifluoromethylphenyl)-4,4-dimethyl-2,6-dioxocyclohexanecarboxamide;
N-(4-fluorophenyl)-4,4-dimethyl-2,6-dioxocyclohexanecarboxamide;
N-(3-trifluoromethoxyphenyl)-4,4-dimethyl-2,6-dioxocyclohexanecarboxamide;
N-(3,4-difluorophenyl)-4,4-dimethyl-2,6-dioxo-cyclohexanecarboxamide;
N-[4-chloro-2-(trifluoromethyl)phenyl]-4,4-dimethyl-2,6-dioxocyclohexanecarboxamide;
4,4-dimethyl-N-(2-nitrophenyl)-2,6-dioxocyclohexanecarboxamide;
4,4-dimethyl-N-(4-nitrophenyl)-2,6-dioxocyclohexanecarboxamide;
N-[4-chloro-3-(trifluoromethyl)phenyl]-4,4-dimethyl-2,6-dioxocyclohexanecarboxamide;
N-(3-chlorophenyl)-4,4-dimethyl-2,6-dioxocyclohexanecarboxamide;
N-(3-trifluoromethylphenyl)-4-ethyl-4-methyl-2,6-dioxocyclohexanecarboxamide;
N-(4-chloro-3-trifluoromethylphenyl)-4-ethyl-4-methyl-2,6-dioxocyclohexanecarboxamide;
N-(3,4-dichlorophenyl)-4-ethyl-4-methyl-2,6-dioxocyclohexanecarboxamide;
N-(3-chloro-4-fluorophenyl)-4-ethyl-4-methyl-2,6-dioxocyclohexanecarboxamide;
N-(4-trifluoromethylphenyl)-4-ethyl-4-methyl-2,6-dioxocyclohexanecarboxamide;
N-(3,5-bis-trifluoromethylphenyl)-4-ethyl-4-methyl-2,6-dioxocyclohexanecarboxamide;
3-Fluoro-4,4-dimethyl-2,6-dioxo-cyclohexanecarboxylic acid(4-trifluoromethyl-phenyl)-amide;
3-Fluoro-4,4-dimethyl-2,6-dioxo-cyclohexanecarboxylic acid(3,5-dichloro-phenyl)-amide;
3-Fluoro-4,4-dimethyl-2,6-dioxo-cyclohexanecarboxylic acid(3,5-difluoro-phenyl)-amide;
4,4-Dimethyl-2,6-dioxo-3-phenyl-cyclohexanecarboxylic acid(3-trifluoromethyl-phenyl)-amide;
3-Methyl-2,6-dioxo-3-phenyl-cyclohexanecarboxylic acid(3-trifluoromethyl-phenyl)-amide;
3-Methoxymethyl-4,4-dimethyl-2,6-dioxo-cyclohexanecarboxylic acid(3-trifluoromethyl-phenyl)-amide; or
a tautomer thereof, or a pharmaceutically acceptable salt thereof or both.

6. The method of claim 1 wherein at least one compound administered has the formula I, or a tautomer, or a pharmaceutically acceptable salt thereof or both wherein:
Y is CR₁R₂;
W is CR₉R₁₀;
Z is CR₃R₄;
two of R₁, R₂, R₃, R₄, R₉, or R₁₀, attached to the same carbon atom or adjacent carbon atoms, form together with the carbon atom or carbon atoms, a saturated or partially saturated carbocyclic or heterocyclic ring in spiro or fused form having 4 to 8 carbon atoms and zero to two heteroatoms independently selected from N, O, or S; and
the remaining R₁, R₂, R₃, R₄, R₉, or R₁₀ are independently selected from hydrogen, a halogen atom, a hydroxyl group, a C₁ to C₆ alkyl group, a C₁ to C₆ alkoxy group, a C₁ to C₁₂ alkoxyalkyl group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl.

7. The method of claim 6 wherein the compound administered comprises at least one of:
2,4-Dioxo-3-(3-trifluoromethyl-phenylcarbamoyl)-decahydro-naphthalene-1-carboxylic acid ethyl ester;
7,9-dioxo-spiro[4.5]decane-8-carboxylic acid(4-trifluoromethyl-phenyl)-amide;
7,9-dioxo-spiro[4.5]decane-8-carboxylic acid(3,5-bis-trifluoromethyl-phenyl)-amide;
7,9-dioxo-spiro[4.5]decane-8-carboxylic acid(3-fluoro-phenyl)-amide;
7,9-dioxo-spiro[4.5]decane-8-carboxylic acid(4-chloro-3-trifluoromethyl-phenyl)-amide;
7,9-dioxo-spiro[4.5]decane-8-carboxylic acid(3-trifluoromethoxy-phenyl)-amide;
2,4-dioxo-spiro[5.5]undecane-3-carboxylic acid(4-trifluoromethyl-phenyl)-amide;
2,4-dioxo-spiro[5.5]undecane-3-carboxylic acid(3,5-bis-trifluoromethyl-phenyl)-amide;
2,4-dioxo-spiro[5.5]undecane-3-carboxylic acid(3-chloro-phenyl)-amide;
2,4-dioxo-spiro[5.5]undecane-3-carboxylic acid(3,4-dichloro-phenyl)-amide;
2,4-dioxo-spiro[5.5]undecane-3-carboxylic acid(4-hydroxy-phenyl)-amide; or
a tautomer thereof, or a pharmaceutically acceptable salt thereof or both.

8. The method of claim 1 wherein at least one compound administered has the formula I, or a tautomer, or a pharmaceutically acceptable salt thereof or both, wherein i) Y is O or S; or ii) W or Z or both are O.

9. The method of claim 8 wherein the compound administered is selected from at least one of:
3,5-dioxo-tetrahydrothiopyran-4-carboxylic acid(4-chloro-3-trifluoromethyl-phenyl)-amide;
3,5-dioxo-tetrahydrothiopyran-4-carboxylic acid(3,4-dichloro-phenyl)-amide;
3,5-dioxo-tetrahydrothiopyran-4-carboxylic acid(3,5-bis-trifluoromethyl-phenyl)-amide;
3,5-dioxo-tetrahydrothiopyran-4-carboxylic acid(4-nitro-phenyl)-amide;
3,5-dioxo-tetrahydrothiopyran-4-carboxylic acid(4-fluoro-3-trifluoromethoxy-phenyl)-amide;
3,5-dioxo-tetrahydrothiopyran-4-carboxylic acid(3-hydroxy-phenyl)-amide;
3,5-dioxo-tetrahydro-pyran-4-carboxylic acid(4-chloro-3-trifluoromethyl-phenyl)-amide;
3,5-dioxo-tetrahydro-pyran-4-carboxylic acid(3,4-dichloro-phenyl)-amide;
3,5-dioxo-tetrahydro-pyran-4-carboxylic acid(4-trifluoromethyl-phenyl)-amide;
3,5-dioxo-tetrahydro-pyran-4-carboxylic acid(3-nitro-phenyl)-amide;
3,5-dioxo-tetrahydro-pyran-4-carboxylic acid(4-trifluoromethoxyl-phenyl)-amide;
3,5-dioxo-tetrahydro-pyran-4-carboxylic acid(4-hydroxy-phenyl)-amide;
N-[4-chloro-3-(trifluoromethyl)phenyl]-6,6-dimethyl-2,4-dioxotetrahydro-2H-pyran-3-carboxamide;
N-[4-(trifluoromethyl)phenyl]-6,6-dimethyl-2,4-dioxotetrahydro-2H-pyran-3-carboxamide;
N-[3-(trifluoromethyl)phenyl]-6,6-dimethyl-2,4-dioxotetrahydro-2H-pyran-3-carboxamide;
N-(3-nitrophenyl)-6,6-dimethyl-2,4-dioxotetrahydro-2H-pyran-3-carboxamide;
N-(3,4-dichlorophenyl)-6,6-dimethyl-2,4-dioxotetrahydro-2H-pyran-3-carboxamide;
N-[3-(trifluoromethyl)phenyl]-5,5-dimethyl-2,4-dioxotetrahydro-2H-pyran-3-carboxamide;
N-[4-(trifluoromethyl)phenyl]-5,5-dimethyl-2,4-dioxotetrahydro-2H-pyran-3-carboxamide;
N-[4-chloro-3-(trifluoromethyl)phenyl]-5,5-dimethyl-2,4-dioxotetrahydro-2H-pyran-3-carboxamide;
7,9-Dioxo-6,10-dioxa-spiro[4.5]decane-8-carbothioic acid(4-chloro-3-trifluoromethyl-phenyl)-amide; or
a tautomer thereof, a pharmaceutically acceptable salt thereof, or both.

10. The method of claim 1 wherein the condition treated is selected from at least one of convulsions, epilepsy, episodic ataxia, myokymia, neonatalconvulsions, cerebral ischemia, cerebral palsy, stroke, traumatic brain injury, traumatic spinal cord injury, asphyxia, anoxia or prolonged cardiac surgery.

11. The method of claim 1 wherein the condition treated is an anxiety disorder.

12. The method of claim 1 wherein the condition treated is a glutamate-related disease selected from hypoglycemia, Parkinson's disease, Huntingdon's disease, Alzheimer's disease, amyotrophic lateral sclerosis, or AIDS related dementia.

13. The method of claim 1 wherein the condition treated is pain.

14. A method of treating one or more conditions in a mammal associated with neuronal hyperexcitability comprising administering to a mammal a pharmaceutically effective amount of at least one compound of formula I, or a tautomer thereof, or a pharmaceutically acceptable salt thereof, or both: 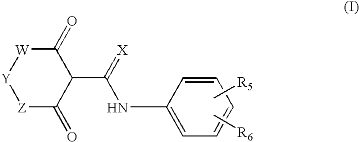
wherein:
Y is CR₁R₂, S or O;
W is CR₉R₁₀ or O;
Z is CR₃R₄ or O; with the proviso that when Y is O or S, W is CR₉R₁₀ and Z is CR₃R₄, and with the proviso that when W or Z or both are O, Y is CR₁R₂;
X is O or S;
R₁, R₂, R₃, R₄, R₉, and R₁₀ are independently selected from hydrogen, a halogen atom, a hydroxyl group, a C₁ to C₆ alkyl group, a C₁ to C₆ alkoxy group, a C₁ to C₁₂ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, or alkanesulfonyl; or two of R₁, R₂, R₃R₄, R₉, or R₁₀, attached to the same carbon atom or adjacent carbon atoms, may form together with the carbon atom or carbon atoms, in spiro or fused form, a carbocyclic or heterocyclic ring having 3 to 8 carbon atoms and zero to two heteroatoms independently selected from N, O, or S;
R₅ and R₆ are independently selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl; and
R₇ and R₈ are independently selected from hydrogen or a C₁ to C₆ alkyl group;
wherein any moiety containing an alkyl, alkenyl, carbocyclic, heterocyclic, cycloalkyl, heterocycloalkyl, aryl, or heteroaryl group may optionally be substituted with one to three substituents independently selected from a halogen atom, CN, NO₂ or hydroxyl group, a C₁-C₆ alkyl group, a C₁-C₆ alkoxy group, a C₁-C₄ perhalogenated alkyl group or C₁-C₄ perhalogenated alkoxy group.

15. The method of claim 14 wherein the condition treated is selected from at least one of convulsions, epilepsy, episodic ataxia, myokymia, neonatal convulsions, cerebral ischemia, cerebral palsy, stroke, traumatic brain injury, traumatic spinal cord injury, asphyxia, anoxia or prolonged cardiac surgery.

16. A method of treating conditions in a mammal related to release of glutamate in the mammal comprising administering to a mammal a pharmaceutically effective amount of at least one compound of formula I, or a tautomer thereof, or a pharmaceutically acceptable salt thereof, or both: 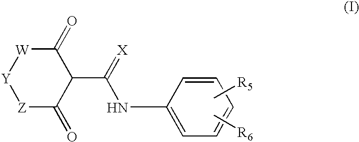
wherein:
Y is CR₁R₂, S or O;
W is CR₉R₁₀ or O;
Z is CR₃R₄ or O; with the proviso that when Y is O or S, W is CR₉R₁₀ and Z is CR₃R₄, and with the proviso that when W or Z or both are O, Y is CR₁R₂;
X is O or S;
R₁, R₂, R₃, R₄, R₉, and R₁₀ are independently selected from hydrogen, a halogen atom, a hydroxyl group, a C₁ to C₆ alkyl group, a C₁ to C₆ alkoxy group, a C₁ to C₁₂ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, or alkanesulfonyl; or two of R₁, R₂, R₃, R₄, R₉, or R₁₀, attached to the same carbon atom or adjacent carbon atoms, may form together with the carbon atom or carbon atoms, in spiro or fused form, a carbocyclic or heterocyclic ring having 3 to 8 carbon atoms and zero to two heteroatoms independently selected from N, O, or S;
R₅ and R₆ are independently selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl; and
R₇ and R₈ are independently selected from hydrogen or a C₁ to C₆ alkyl group;
wherein any moiety containing an alkyl, alkenyl, carbocyclic, heterocyclic, cycloalkyl, heterocycloalkyl, aryl, or heteroaryl group may optionally be substituted with one to three substituents independently selected from a halogen atom, CN, NO₂ or hydroxyl group, a C₁-C₆ alkyl group, a C₁-C₆ alkoxy group, a C₁-C₄ perhalogenated alkyl group or C₁-C₄ perhalogenated alkoxy group.

17. The method of claim 16 wherein the condition treated is selected from Alzheimer's disease, Parkinson's disease, hypoglycemia, Huntingdon's disease, amyotrophic lateral sclerosis, or AIDS related dementia.

18. A method of treating a condition selected from pain, epilepsy, or anxiety disorder, in a mammal comprising administering to a mammal a pharmaceutically effective amount of at least one compound of formula I, or a tautomer thereof, or a pharmaceutically acceptable salt thereof, or both: 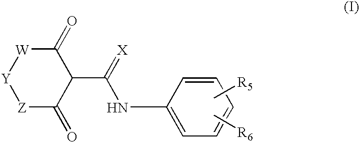
wherein:
Y is CR₁R₂, S or O;
W is CR₉R₁₀ or O;
Z is CR₃R₄ or O; with the proviso that when Y is O or S, W is CR₉R₁₀ and Z is CR₃R₄, and with the proviso that when W or Z or both are O, Y is CR₁R₂;
X is O or S;
R₁, R₂, R₃, R₄, R₉, and R₁₀ are independently selected from hydrogen, a halogen atom, a hydroxyl group, a C₁ to C₆ alkyl group, a C₁ to C₆ alkoxy group, a C₁ to C₁₂ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, or alkanesulfonyl; or two of R₁, R₂, R₃, R₄, R₉, or R₁₀, attached to the same carbon atom or adjacent carbon atoms, may form together with the carbon atom or carbon atoms, in spiro or fused form, a carbocyclic or heterocyclic ring having 3 to 8 carbon atoms and zero to two heteroatoms independently selected from N, O, or S;
R₅ and R₆ are independently selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl; and
R₇ and R₈ are independently selected from hydrogen or a C₁ to C₆ alkyl group;
wherein any moiety containing an alkyl, alkenyl, carbocyclic, heterocyclic, cycloalkyl, heterocycloalkyl, aryl, or heteroaryl group may optionally be substituted with one to three substituents independently selected from a halogen atom, CN, NO₂ or hydroxyl group, a C₁-C₆ alkyl group, a C₁-C₆ alkoxy group, a C₁-C₄ perhalogenated alkyl group or C₁-C₄ perhalogenated alkoxy group.

19. A pharmaceutical composition comprising:
a) a pharmaceutically effective amount of at least one compound of formula I, or a tautomer thereof, or a pharmaceutically acceptable salt thereof, or both: 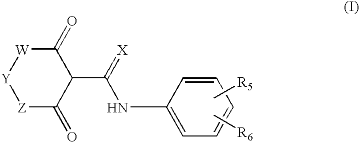
wherein:
Y is CR₁R₂, S or O;
W is CR₉R₁₀ or O;
Z is CR₃R₄ or O; with the proviso that when Y is O or S, W is CR₉R₁₀ and Z is CR₃R₄, and with the proviso that when W or Z or both are O, Y is CR₁R₂;
X is O or S;
R₁, R₂, R₃, R₄, R₉, and R₁₀ are independently selected from hydrogen, a halogen atom, a hydroxyl group, a C₁ to C₆ alkyl group, a C₁ to C₆ alkoxy group, a C₁ to C₁₂ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, or alkanesulfonyl; or two of R₁, R₂, R₃, R₄, R₉, or R₁₀, attached to the same carbon atom or adjacent carbon atoms, may form together with the carbon atom or carbon atoms, in spiro or fused form, a carbocyclic or heterocyclic ring having 3 to 8 carbon atoms and zero to two heteroatoms independently selected from N, O, or S;
R₅ and R₆ are independently selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇RF, CO₂R₇, COR₇, or alkanesulfonyl; and
R₇ and R₈ are independently selected from hydrogen or a C₁ to C₆ alkyl group;
wherein any moiety containing an alkyl, alkenyl, carbocyclic, heterocyclic, cycloalkyl, heterocycloalkyl, aryl, or heteroaryl group may optionally be substituted with one to three substituents independently selected from a halogen atom, CN, NO₂ or hydroxyl group, a C₁-C₆ alkyl group, a C₁-C₆ alkoxy group, a C₁-C₄ perhalogenated alkyl group or C₁-C₄ perhalogenated alkoxy group; and
(b) at least one pharmaceutically acceptable carrier.

20. A compound of formula II, or a tautomer thereof, or a pharmaceutically acceptable salt thereof, or both: 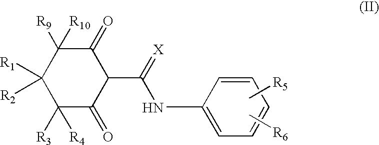
wherein:
two of R₁, R₂, R₃, R₄, R₉, or R₁₀, attached to the same carbon atom form together with the carbon atom, a carbocyclic or heterocyclic ring, in spiro form, having 3 to 8 carbon atoms and zero to two heteroatoms independently selected from N, O, or S, and the remaining R₁, R₂, R₃, R₄, R₉, or R₁₀ are selected from hydrogen, a halogen atom, a hydroxyl group, a C₁ to C₆ alkyl group, a C₁ to C₆ alkoxy group, a C₁ to C₁₂ alkoxyalkyl group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl;
R₅ and R₆ are independently selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl; and
R₇ and R₈ are independently selected from hydrogen or a C₁ to C₆ alkyl group;
wherein any moiety containing an alkyl, alkenyl, carbocyclic, heterocyclic, cycloalkyl, heterocycloalkyl, aryl, or heteroaryl group may optionally be substituted with one to three substituents independently selected from a halogen atom, CN, NO₂ or hydroxyl group, a C₁-C₆ alkyl group, a C₁-C₆ alkoxy group, a C₁-C₄ perhalogenated alkyl group or C₁-C₄ perhalogenated alkoxy group.

21. The compound of claim 20 wherein the compound is selected from
7,9-dioxo-spiro[4.5]decane-8-carboxylic acid(4-trifluoromethyl-phenyl)-amide;
7,9-dioxo-spiro[4.5]decane-8-carboxylic acid(3,5-bis-trifluoromethyl-phenyl)-amide;
7,9-dioxo-spiro[4.5]decane-8-carboxylic acid(3-fluoro-phenyl)-amide;
7,9-dioxo-spiro[4.5]decane-8-carboxylic acid(4-chloro-3-trifluoromethyl-phenyl)-amide;
7,9-dioxo-spiro[4.5]decane-8-carboxylic acid(3-trifluoromethoxy-phenyl)-amide;
2,4-dioxo-spiro[5.5]undecane-3-carboxylic acid(4-trifluoromethyl-phenyl)-amide;
2,4-dioxo-spiro[5.5]undecane-3-carboxylic acid(3,5-bis-trifluoromethyl-phenyl)-amide;
2,4-dioxo-spiro[5.5]undecane-3-carboxylic acid(3-chloro-phenyl)-amide;
2,4-dioxo-spiro[5.5]undecane-3-carboxylic acid(3,4-dichloro-phenyl)-amide; or
2,4-dioxo-spiro[5.5]undecane-3-carboxylic acid(4-hydroxy-phenyl)-amide; or
a tautomer thereof, or a pharmaceutically acceptable salt thereof, or both.

22. A compound of formula IV or V, or a tautomer thereof or a pharmaceutically acceptable salt thereof, or both 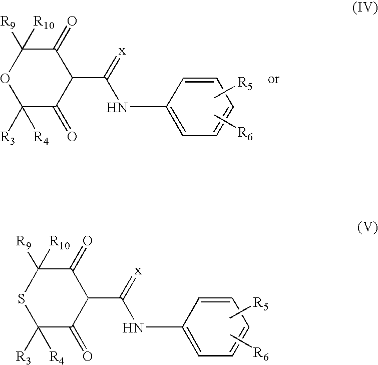
wherein:
X is O or S;
R₃, R₄, R₉ and R₁₀ are independently selected from hydrogen, a halogen atom, a hydroxyl group, a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, alkanesulfonyl; or one of R₉ and R₁₀, or R₃ and R₄ may form together with the carbon atom a carbocyclic or heterocyclic ring in spiro form having 3 to 8 carbon atoms and zero to two heteroatoms independently selected from N, O, or S;
R₅ and R₆ are independently selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl; and
R₇ and R₈ are independently selected from hydrogen or a C₁ to C₆ alkyl group;
wherein any moiety containing an alkyl, alkenyl, carbocyclic, heterocyclic, cycloalkyl, heterocycloalkyl, aryl, or heteroaryl group may optionally be substituted with one to three substituents independently selected from a halogen atom, CN, NO₂ or hydroxyl group, a C₁-C₆ alkyl group, a C₁-C₆ alkoxy group, a C₁-C₄ perhalogenated alkyl group or C₁-C₄ perhalogenated alkoxy group.

23. The compound of claim 22 wherein the compound is selected from
3,5-dioxo-tetrahydrothiopyran-4-carboxylic acid(4-chloro-3-trifluoromethyl-phenyl)-amide;
3,5-dioxo-tetrahydrothiopyran-4-carboxylic acid(3,4-dichloro-phenyl)-amide;
3,5-dioxo-tetrahydrothiopyran-4-carboxylic acid(3,5-bis-trifluoromethyl-phenyl)-amide;
3,5-dioxo-tetrahydrothiopyran-4-carboxylic acid(4-nitro-phenyl)-amide;
3,5-dioxo-tetrahydrothiopyran-4-carboxylic acid(4-fluoro-3-trifluoromethoxy-phenyl)-amide;
3,5-dioxo-tetrahydrothiopyran-4-carboxylic acid(3-hydroxy-phenyl)-amide;
3,5-dioxo-tetrahydro-pyran-4-carboxylic acid(4-chloro-3-trifluoromethyl-phenyl)-amide;
3,5-dioxo-tetrahydro-pyran-4-carboxylic acid(3,4-dichloro-phenyl)-amide;
3,5-dioxo-tetrahydro-pyran-4-carboxylic acid(4-trifluoromethyl-phenyl)-amide;
3,5-dioxo-tetrahydro-pyran-4-carboxylic acid(3-nitro-phenyl)-amide;
3,5-dioxo-tetrahydro-pyran-4-carboxylic acid(4-trifluoromethoxyl-phenyl)-amide; or
3,5-dioxo-tetrahydro-pyran-4-carboxylic acid(4-hydroxy-phenyl)-amide; or
a tautomer thereof, or a pharmaceutically acceptable salt thereof, or both.

24. A compound of formula VII or a tautomer thereof, or a pharmaceutically acceptable salt thereof, or both: 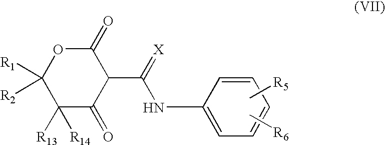
wherein:
X is S or O;
R₁, R₂, R₁₃ and R₁₄, are independently selected from hydrogen, a halogen atom, a hydroxyl group, a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group, a C₂ to C₇ alkenyl group, C₃ to C₈ cycloalkyl group, C₅ to C₇ aryl group, C₆ to C₁₃ arylalkyl group of 1 to 6 carbon atoms in the alkyl chain, a C₅ to C₇ aryloxy group, C₆ to C₁₃ arylalkyloxy group of 1 to 6 carbon atoms in the alkyl chain, CN, NO₂, NR₇R₈, CO₂R₇, COR₇, alkanesulfonyl; with the proviso that at least one of i) R₁ and R₂, or ii) R₁₃ and R₁₄ are not hydrogen and are independently selected from a C₁ to C₆ alkyl group, a C₁ to C₆ alkoxy group, a C₁ to C₆ alkoxyalkyl group, a C₁ to C₄ perhalogenated alkyl group, a C₁ to C₄ perhalogenated alkoxy group, or one of i) R₁ and R₂, or ii) R₁₃ and R₁₄, together with the carbon atom to which they are attached form a carbocyclic or heterocyclic ring in spiro form having 3 to 8 carbon atoms and zero to two heteroatoms independently selected from N, O, or S;
R₅ and R₆ are independently selected from hydrogen, a halogen atom, a hydroxyl group, a C₁ to C₆ alkyl group, a C₁ to C₆ alkoxy group, a C₁ to C₄ perhalogenated alkyl group, a C₁ to C₄ perhalogenated alkoxy group, a C₂ to C₇ alkenyl group, CN, NO₂, NR₇R₈, CO₂R₇, COR₇, or alkanesulfonyl; and
R₇ and R₈ are independently selected from hydrogen or a C₁ to C₆ alkyl group;
wherein any moiety containing an alkyl, alkenyl, carbocyclic, heterocyclic, cycloalkyl, heterocycloalkyl, aryl, or heteroaryl group may optionally be substituted with one to three substituents independently selected from a halogen atom, CN, NO₂ or hydroxyl group, a C₁-C₆ alkyl group, a C₁-C₆ alkoxy group, a C₁-C₄ perhalogenated alkyl group or C₁-C₄ perhalogenated alkoxy group.

25. The compound of claim 24 wherein the compound is selected from:
N-[4-chloro-3-(trifluoromethyl)phenyl]-6,6-dimethyl-2,4-dioxotetrahydro-2H-pyran-3-carboxamide;
N-[4-(trifluoromethyl)phenyl]-6,6-dimethyl-2,4-dioxotetrahydro-2H-pyran-3-carboxamide;
N-[3-(trifluoromethyl)phenyl]-6,6-dimethyl-2,4-dioxotetrahydro-2H-pyran-3-carboxamide;
N-(3-nitrophenyl)-6,6-dimethyl-2,4-dioxotetrahydro-2H-pyran-3-carboxamide;
N-(3,4-dichlorophenyl)-6,6-dimethyl-2,4-dioxotetrahydro-2H-pyran-3-carboxamide;
N-[3-(trifluoromethyl)phenyl]-5,5-dimethyl-2,4-dioxotetrahydro-2H-pyran-3-carboxamide;
N-[4-(trifluoromethyl)phenyl]-5,5-dimethyl-2,4-dioxotetrahydro-2H-pyran-3-carboxamide;
N-[4-chloro-3-(trifluoromethyl)phenyl]-5,5-dimethyl-2,4-dioxotetrahydro-2H-pyran-3-carboxamide; or
a tautomer thereof, or a pharmaceutically acceptable salt thereof, or both.

26. A compound which is:
7,9-Dioxo-6,10-dioxa-spiro[4.5]decane-8-carbothioic acid(4-chloro-3-trifluoromethyl-phenyl)-amide; or
2,4-Dioxo-3-(3-trifluoromethyl-phenylcarbamoyl)-decahydro-naphthalene-1-carboxylic acid ethyl ester; or
a tautomer thereof, or a pharmaceutically acceptable salt thereof, or both.
